(19) United States
(12) Patent Application Publication
(10) Pub. No.: US 2024/0238294 A1
(43) Pub. Date: Jul. 18, 2024

(54) ANTICANCER THERAPY

(71) Applicant: Boehringer Ingelheim International GmbH, Ingelheim am Rhein (DE)

(72) Inventors: Tetsuya MITSUDOMI, Osaka-Sayama (JP); Takamasa KOGA, Osaka-Sayama (JP); Marco Hans HOFMANN, Vienna (AT); Michael GMACHL, Vienna (AT)

(21) Appl. No.: 18/554,231

(22) PCT Filed: Apr. 7, 2022
(86) PCT No.: PCT/EP2022/059273

§ 371 Date: Oct. 6, 2023

(30) Foreign Application Priority Data
Apr. 9, 2021 (EP) 21167637.4

(51) Int. Cl. A61K 31/517; A61K 31/519; A61P 35/00; G01N 33/574

(52) U.S. Cl. CPC A61K 31/517; A61K 31/519; A61P 35/00; G01N 33/5748

(57) ABSTRACT

The present invention relates to a Son of Sevenless 1 (SOS1) inhibitor and/or a mitogen-activated protein kinase kinase (MEK) inhibitor for use in the treatment and/or prevention of cancer, wherein the SOS1 inhibitor or the MEK inhibitor is administered alone or the SOS1 inhibitor is administered in combination with the MEK inhibitor, wherein the cancer is resistant to treatment with an inhibitor of KRAS G12C.

### FIELD OF THE INVENTION

The present invention relates to a Son of Sevenless 1 (SOS1) inhibitor, or a mitogen-activated protein kinase kinase (MEK) inhibitor, or a combination of a SOS1 inhibitor and a MEK inhibitor for use in the treatment of cancer that is resistant to treatment with an inhibitor of KRAS G12C, particularly of cancer where the cancer cells exhibit a primary KRAS G12C mutation and further a secondary mutation Y96X, preferably Y96D or Y96S.

### BACKGROUND

V-Ki-ras2 Kirsten rat sarcoma viral oncogene homolog (KRAS) is one of the human RAS genes coding for a RAS GTPase. The KRAS protein is a small GTPases that acts as a molecular switch in the growth factor signalling pathway affecting diverse cellular processes such as proliferation, metabolism and growth. KRAS switches between a GDP-bound inactive conformation and a GTP-bound active conformation. The binding of guanine nucleotide exchange factors (GEFs) such as SOS1 promotes the release of GDP from RAS-family proteins, enabling GTP binding and resulting in an active form which activates downstream pathways.

SOS1 (Son of Sevenless 1) is a human homologue of the originally identified Drosophila protein Son of Sevenless. SOS1 is critically involved in the activation of RAS-family protein signaling in cancer. Selective pharmacological inhibition of the binding of the catalytic site of SOS1 to RAS-family proteins can prevent SOS1-mediated activation of RAS-family proteins to the GTP-bound form, and SOS1 inhibitors inhibit signaling in cells downstream of RAS-family proteins. In cancer cells associated with dependence on RAS-family proteins SOS1 inhibitors provide anti-cancer efficacy. A combination of a SOS1 inhibitor with an inhibitor of MEK (mitogen-activated protein kinase kinase) for use in the treatment of cancer is known from WO 2020/254451 A1.

KRAS is a gene that is commonly mutated in cancer. KRAS mutations, such as amino acids G12, G13, Q61, A146, are found in a variety of human cancers including lung cancer, colorectal cancer and pancreatic cancer. Most of the KRAS mutations in non-small cell lung cancer (NSCLC) occur at codon 12 and approximately half of them are glycine to cysteine substitution (G12C). Despite its high frequency, the development of targeted therapy against KRAS mutated cancer has long been unsatisfactory. Recently, several drugs have been created that can inhibit the function of KRAS proteins that have a G12C mutation. Several KRAS G12C inhibitors have reached clinical trials, including adagrasib and sotorasib, which have demonstrated promising results in metastatic non-small cell lung cancer carrying a KRAS G12C mutation.

Generally, in almost all solid tumors responsive to target therapies, acquired on-target drug resistance inevitably develops, leading to clinical relapse. Thus, acquisition of secondary mutations of the target gene (on-target mechanisms) resulting in acquired resistance inevitably will occur in targeted therapies using adagrasib and sotorasib. The mechanisms of resistance to KRAS G12C inhibitors and possible targets of combination therapy are currently under discussion (Dunnett-Kane et al., Cancers, 2021, 13(1), 151) and both, adagrasib and sotorasib, are already tested in combination with SHP2 (Src homology region 2 domain-containing phosphatase-2) inhibitors in early-phase clinical trials (Hata et al., Nat. Med., 2020, 26, 169-170).

However, so far no data was reported describing secondary mutations in KRAS causing acquired resistance to KRAS G12C inhibitors. Still, secondary mutations in KRAS that would confer acquired resistance to sotorasib and adagrasib are to be expected and possible strategies to overcome this resistance are needed.

It is thus an object of the present invention to provide inhibitors that allow for a secondary treatment option for cancers with acquired resistance to KRAS G12C inhibitors.

### SUMMARY

In cell line models for acquired on-target resistance against sotorasib and adagrasib, clones with secondary mutations Y96D or Y96S (Y96D/S) were found to be resistant to KRAS G12C inhibitors sotorasib and adagrasib. A SOS1 inhibitor, such as BI-3406, and a combination of the SOS1 inhibitor and a MEK inhibitor, such as trametinib, showed potent activity against cells harboring a primary G12C mutation plus a secondary Y96D or Y96S mutation. This provides an option for a second-line therapy to overcome an acquired resistance caused by secondary KRAS mutations as are expected to emerge in the course of treatment with sotorasib and adagrasib.

According to a first aspect is provided a Son of Sevenless 1 (SOS1) inhibitor and/or a mitogen-activated protein kinase kinase (MEK) inhibitor for use in the treatment and/or prevention of cancer, wherein the SOS1 inhibitor or the MEK inhibitor is administered alone or the SOS1 inhibitor is administered in combination with the MEK inhibitor, wherein the cancer is resistant to treatment with an inhibitor of KRAS G12C.

In an embodiment, the cancer has become resistant to treatment with an inhibitor of KRAS G12C after earlier treatment with an inhibitor of KRAS G12C.

In an embodiment, the cancer is resistant to treatment with sotorasib (AMG 510) and/or adagrasib (MRTX 849).

In preferred embodiments, the cancer has become resistant to treatment with sotorasib (AMG 510) and/or adagrasib (MRTX 849) after earlier treatment with sotorasib (AMG 510) and/or adagrasib (MRTX 849).

In an embodiment, the cancer cells of the cancer exhibit a primary KRAS G12C mutation and further exhibit a secondary mutation Y96X, preferably selected from the group consisting of Y96D and Y96S.

In an embodiment, the cancer cells of the cancer have been determined to exhibit a primary KRAS G12C mutation and to further exhibit a secondary mutation Y96X, preferably selected from the group consisting of Y96D and Y96S.

In embodiments, the SOS1 inhibitor is selected from the group consisting of the following compounds or a pharmaceutically acceptable salt thereof:

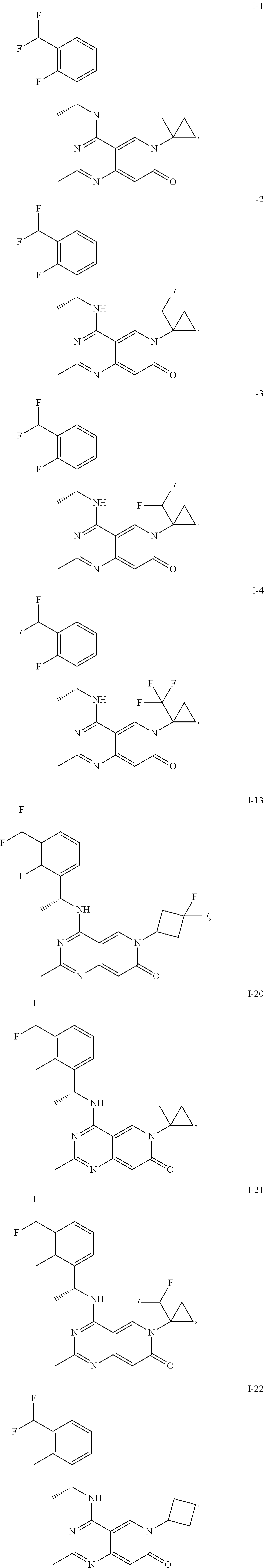

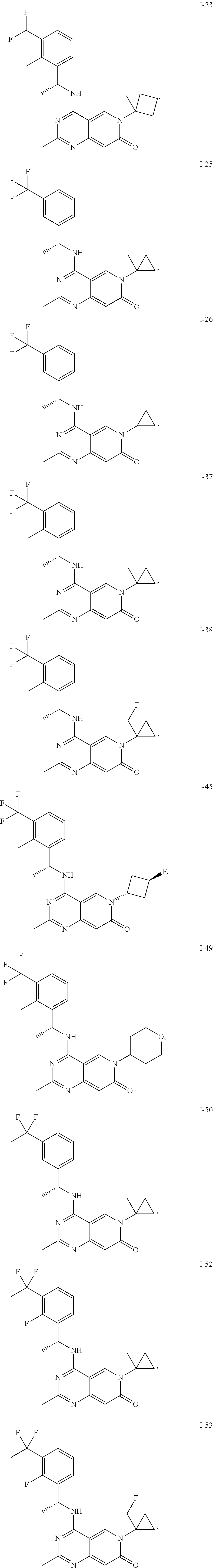

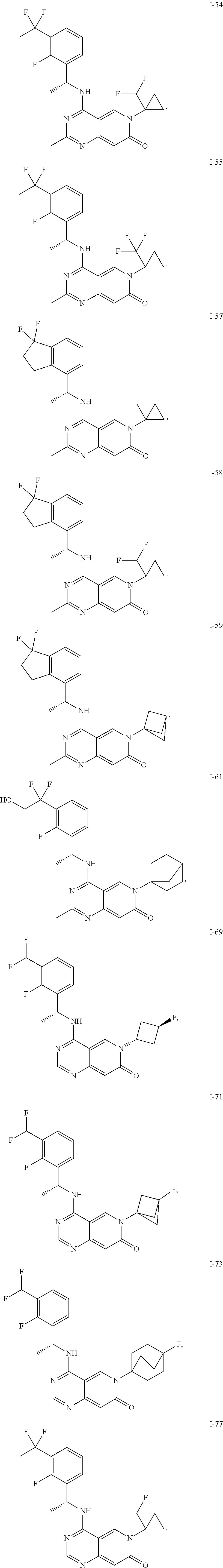

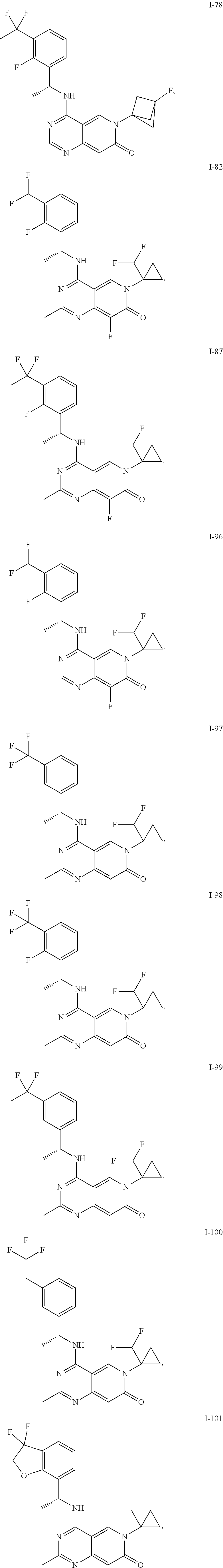

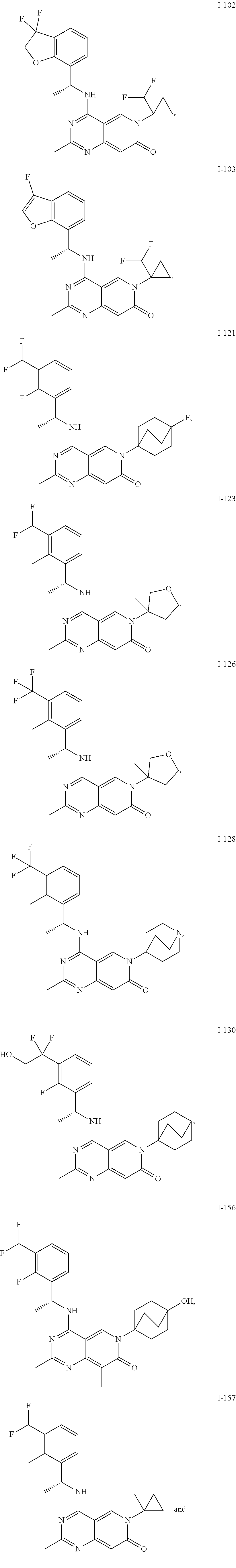

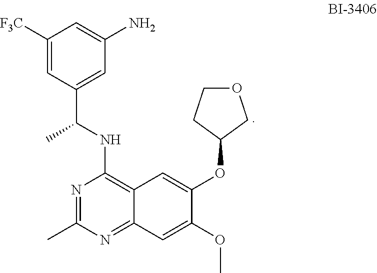 [SMILES: COc1cc2nc(C)nc(N[C@H](C)c3cc(N)cc(C(F)(F)F)c3)c2cc1O[C@H]1CCOC1]

In embodiments, the MEK inhibitor is selected from the group consisting of trametinib, cobimetinib, binimetinib, selumetinib, refametinib and the following compounds, or a pharmaceutically acceptable salt thereof:

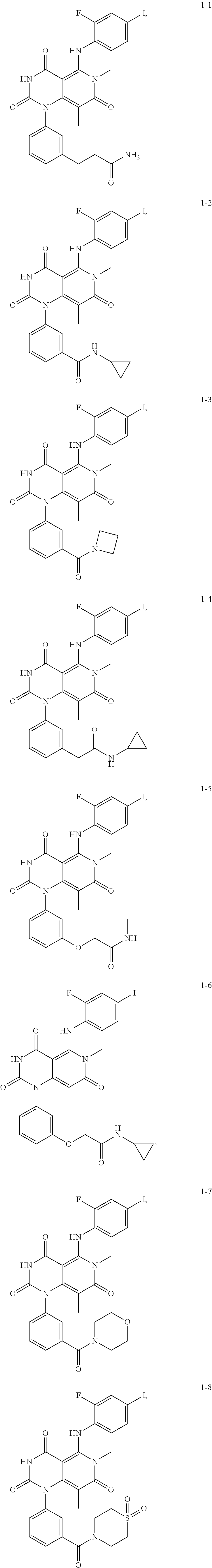

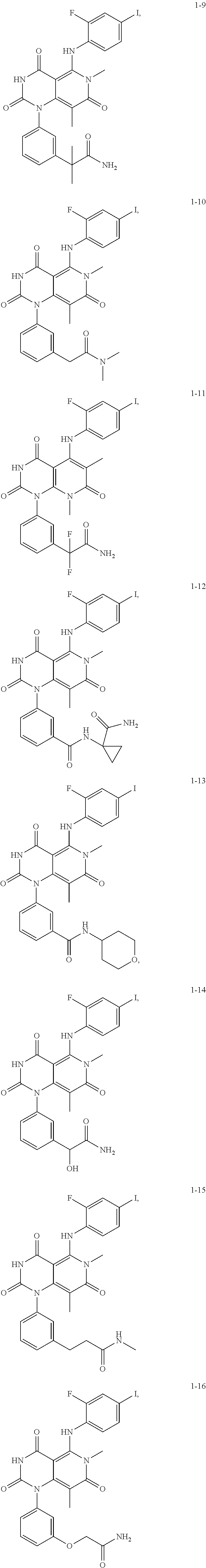

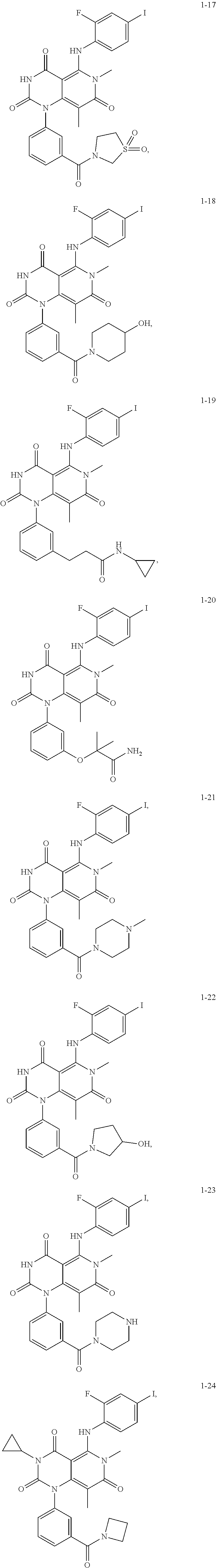

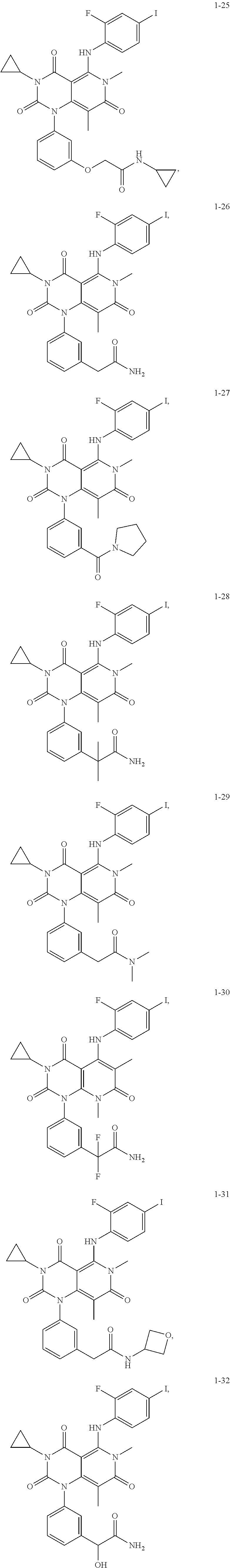

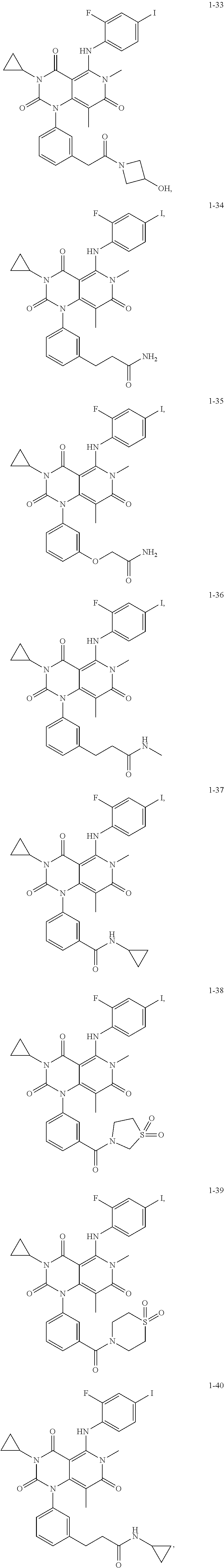

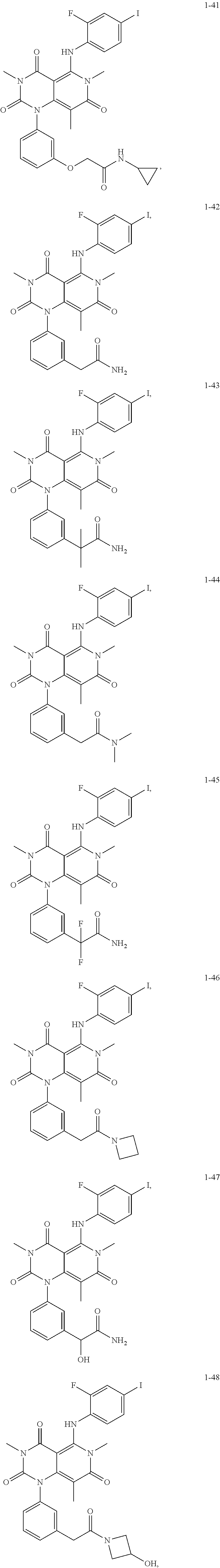

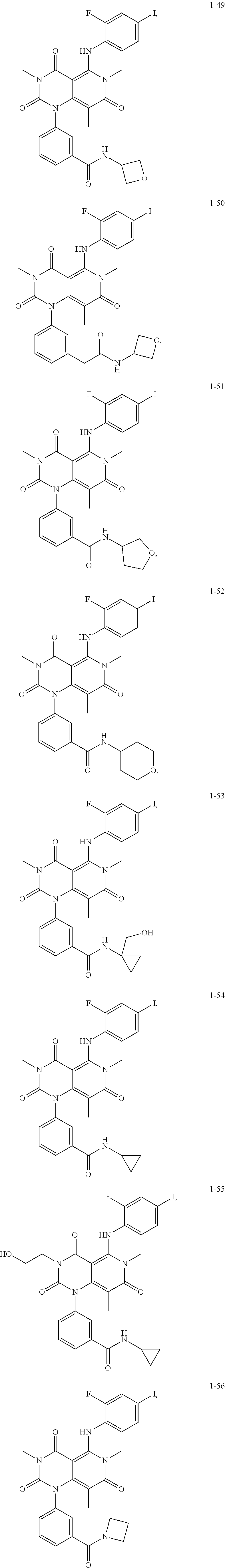

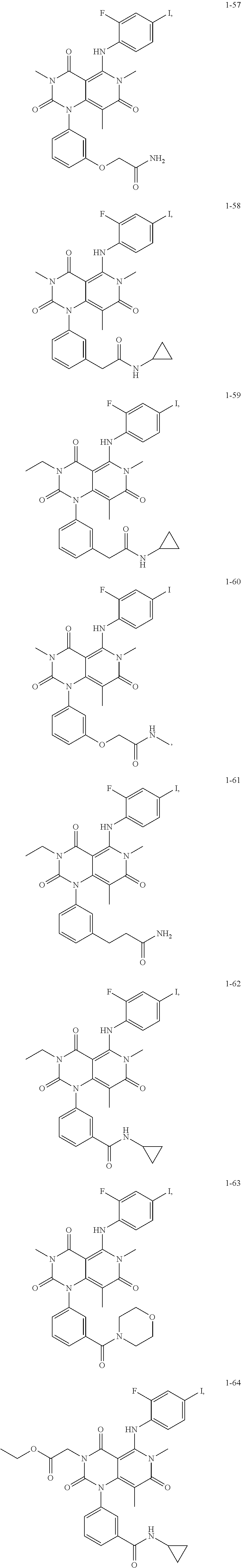

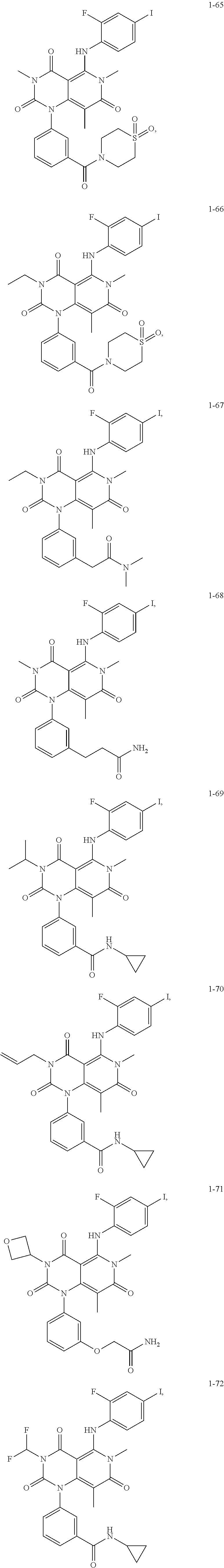

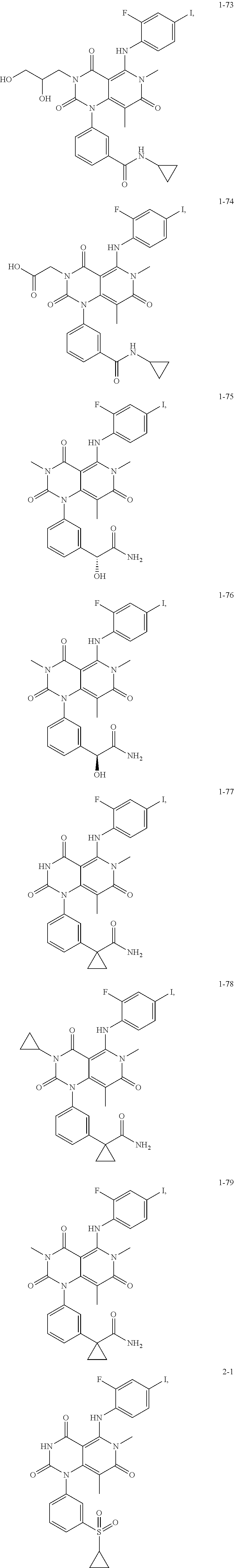

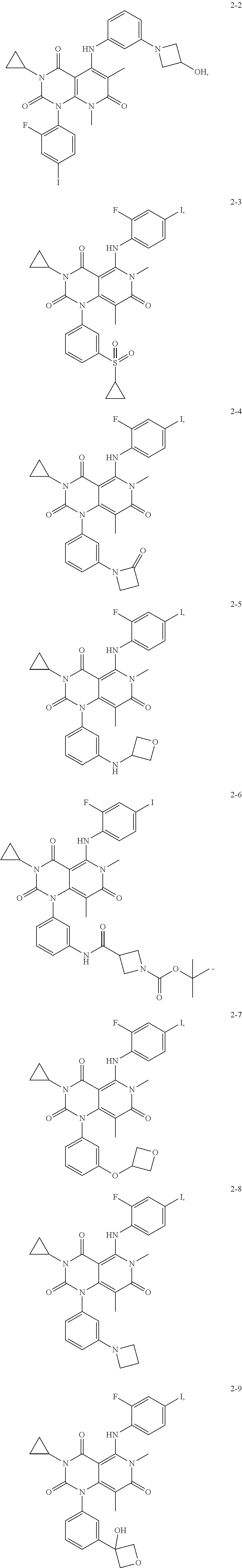

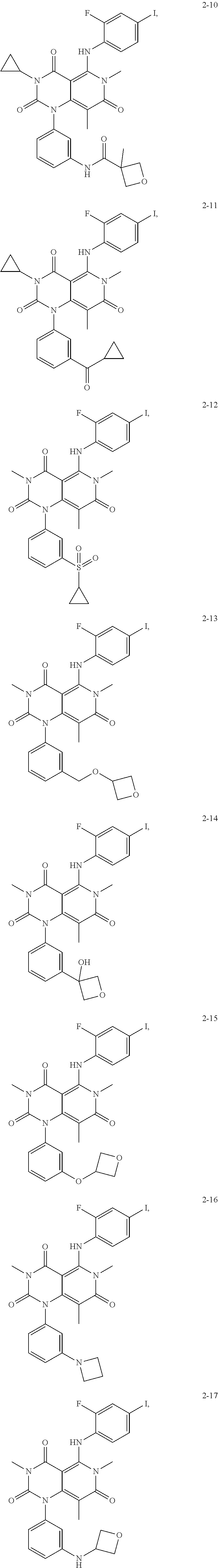

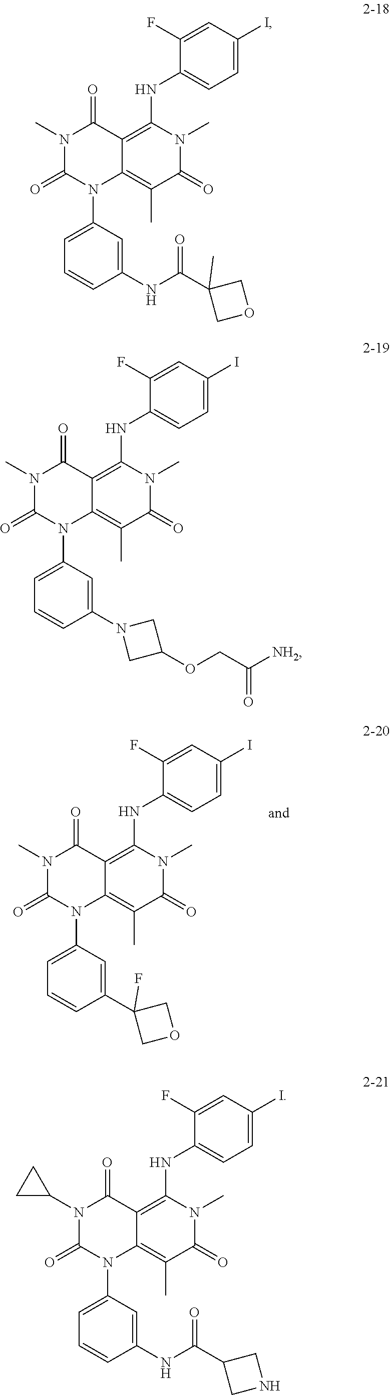

In embodiments, the cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

Another aspect relates to a pharmaceutical composition comprising as active ingredient a SOS1 inhibitor. Another aspect relates to a pharmaceutical composition comprising as active ingredient a MEK inhibitor. Another aspect relates to a pharmaceutical combination comprising as active ingredients a SOS1 inhibitor and a MEK inhibitor, for use in the treatment and/or prevention of cancer, wherein the cancer is resistant to treatment with an inhibitor of KRAS G12C.

Another aspect relates to a kit comprising in one or more containers:
(i) a (first) pharmaceutical composition or dosage form comprising a SOS1 inhibitor, and, optionally, pharmaceutically acceptable carriers, excipients and/or vehicles; and/or
(ii) a (second) pharmaceutical composition or dosage form comprising a MEK inhibitor, and, optionally, pharmaceutically acceptable carriers, excipients and/or vehicles; and
(iii) optionally a package insert comprising instructions,
for use in the treatment and/or prevention of cancer, wherein the cancer is resistant to treatment with an inhibitor of KRAS G12C.

Another aspect relates to a method of treating and/or preventing cancer, the method comprising administering to a patient a therapeutically effective amount of a SOS1 inhibitor or a therapeutically effective amount of a MEK inhibitor or a therapeutically effective amount of a SOS1 inhibitor in combination with a therapeutically effective amount of a MEK inhibitor, wherein the cancer is resistant to treatment with an inhibitor of KRAS G12C.

Another aspect relates to a use of a SOS1 inhibitor and/or a MEK inhibitor for the manufacture of a medicament for use in the treatment and/or prevention of cancer, wherein the SOS1 inhibitor or the MEK inhibitor is to be used alone or the SOS1 inhibitor is to be used in combination with the MEK inhibitor, wherein the cancer is resistant to treatment with an inhibitor of KRAS G12C.

In an embodiment of all aspects disclosed herein, particularly of the pharmaceutical composition or combination, the kit, the methods, or the uses, the cancer has become resistant to treatment with an inhibitor of KRAS G12C after earlier treatment with an inhibitor of KRAS G12C.

In an embodiment of all aspects disclosed herein, particularly of the pharmaceutical composition or combination, the kit, the methods, or the uses, the SOS1 inhibitor is a compound as defined herein or a pharmaceutically acceptable salt thereof.

In an embodiment of all aspects disclosed herein, particularly of the pharmaceutical composition or combination, the kit, the methods, or the uses, the MEK inhibitor is a compound as defined herein or a pharmaceutically acceptable salt thereof.

In an embodiment of all aspects disclosed herein, particularly of the pharmaceutical composition or combination, the kit, the methods, or the uses, the cancer is resistant to treatment with sotorasib (AMG 510) and/or adagrasib (MRTX 849).

In preferred embodiments of all aspects disclosed herein, particularly of the pharmaceutical composition or combination, the kit, the methods, or the uses, the cancer has become resistant to treatment with sotorasib (AMG 510) and/or adagrasib (MRTX 849) after earlier treatment with sotorasib (AMG 510) and/or adagrasib (MRTX 849).

In an embodiment of all aspects disclosed herein, particularly of the pharmaceutical composition or combination, the kit, the methods, or the uses, the cancer cells of the cancer exhibit a primary KRAS G12C mutation and further exhibit a secondary mutation Y96X, preferably selected from the group consisting of Y96D and Y96S.

In preferred embodiments of all aspects disclosed herein, particularly of the pharmaceutical composition or combination, the kit, the methods, or the uses, the cancer cells of the cancer have been determined to exhibit a primary KRAS G12C mutation and to further exhibit a secondary mutation Y96X, preferably selected from the group consisting of Y96D and Y96S.

In embodiments of all aspects disclosed herein, particularly of the pharmaceutical composition or combination, the kit, the methods, or the uses, the cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

Another aspect relates to an in vitro method for detecting or diagnosing that an individual has acquired resistance to treatment with an inhibitor of KRAS G12C and/or is susceptible to treatment with a SOS1 inhibitor or a MEK inhibitor or a combination of a SOS1 inhibitor and a MEK inhibitor, the method comprising the step of:
determining, in a biological sample from an individual, the presence of a KRAS mutation Y96X, preferably selected from the group consisting of Y96D and Y96S, wherein the individual is classified as being resistant to treatment with an inhibitor of KRAS G12C and being susceptible to treatment with a SOS1 inhibitor or a MEK inhibitor or a combination of a SOS1 inhibitor and a MEK inhibitor, based on the detection of the KRAS mutation Y96X, preferably selected from the group consisting of Y96D and Y96S.

Another aspect relates to a use of a KRAS mutation Y96X, preferably selected from the group consisting of Y96D and Y96S as a biomarker for a cancer or cancer cells being resistant to treatment with an inhibitor of KRAS G12C and/or the cancer or cancer cells being susceptible to treatment with a SOS1 inhibitor or a MEK inhibitor or a combination of a SOS1 inhibitor and a MEK inhibitor.

### BRIEF DESCRIPTION OF THE FIGURES

FIG. 1 Growth inhibition curves for engineered Ba/F3 cells harboring KRAS G12C, G12D or G12V. FIG. 1A shows the cells treated with the indicated concentrations of sotorasib for 72 hours. The results are shown as the mean values of three individual experiments. Error bars indicate the standard deviation (SD). FIG. 1B shows growth inhibition curves for engineered Ba/F3 cells harboring KRAS G12C, G12D or G12V treated with the indicated concentrations of adagrasib for 72 hours.

FIG. 2 Growth inhibition curves of Ba/F3 cells harboring KRAS G12C plus secondary mutations derived through ENU mutagenesis. FIG. 2A shows the cells treated with the indicated concentrations of sotorasib for 72 hours. The results are shown as the mean values of three individual experiments. Error bars indicate the standard deviation (SD). FIG. 2B shows growth inhibition curves of Ba/F3 cells harboring KRAS G12C+secondary mutations derived through ENU mutagenesis and treated with the indicated concentrations of adagrasib for 72 hours.

FIG. 3 Growth inhibition assay of Ba/F3 cells harboring KRAS G12C plus secondary mutations both introduced into Ba/F3 cells. FIG. 3A shows the cells treated with the indicated concentrations of sotorasib for 72 hours. The results are shown as the mean values of three individual experiments. Error bars indicate the standard deviation (SD). FIG. 3B shows growth inhibition assay of Ba/F3 cells harboring KRAS G12C plus secondary mutations and treated with the indicated concentrations of adagrasib for 72 hours.

FIG. 4 Growth inhibition assay of H358 cells with KRAS G12C plus secondary mutations introduced into H358 cells. FIG. 4A shows the cells with the indicated concentrations of sotorasib for 72 hours. The results are shown as the mean values of three individual experiments. Error bars indicate the standard deviation (SD). FIG. 4B shows growth inhibition assay of H358 cells with KRAS G12C plus secondary mutations treated with the indicated concentrations of adagrasib for 72 hours.

FIG. 5 Growth inhibition assay of Ba/F3 cells harboring KRAS G12C plus secondary A59S, Y96D or Y96S mutation (all introduced into Ba/F3 cells) treated with the indicated concentrations of BI 3406 for 72 hours in FIG. 5A. The results are shown as the mean values of three individual experiments. Error bars indicate the standard deviation (SD). FIG. 5B shows three-dimensional (3D) growth inhibition assay of H358 cells with KRAS G12C plus secondary A59S, Y96D or Y96S mutation (introduced into H358 cells) treated with the indicated concentrations of BI 3406 for 72 hours. FIG. 5C shows three-dimensional (3D) growth inhibition assay of H358 cells with KRAS G12C plus secondary Y96D or Y96S mutation (introduced into H358 cells) treated with the indicated concentrations of trametinib and 1 μM BI 3406 for 72 hours

### DETAILED DESCRIPTION

### SOS 1 Inhibitor

As used herein, the term “SOS1” refers to the human homologue of the Drosophila protein Son of Sevenless. As used herein, the term “SOS1 inhibitor” refers to a compound that inhibits the binding of SOS1 to KRAS preventing SOS1-mediated activation of KRAS to the GTP-bound form. In an embodiment the SOS1 inhibitor binds to SOS1. SOS1 inhibitors belonging to different compound classes are known.

Preferably, referring to all aspects and embodiments, including methods of treatment, uses, combinations, and compositions, the SOS1 inhibitor is selected from the group consisting of:
SOS1 inhibitors generically described and/or specifically disclosed in WO 2018/115380,
any SOS1 inhibitor I-1 to I-383 as disclosed in WO 2018/115380,
SOS1 inhibitors generically described and/or specifically disclosed in WO 2019/122129,
any SOS1 inhibitor I-1 to I-179 as disclosed in WO 2019/122129,
SOS1 inhibitors generically described and/or specifically disclosed in WO 2020/180768,
any SOS1 inhibitor example 1 to 103 as disclosed in WO 2020/180768,
SOS1 inhibitors generically described and/or specifically disclosed in WO 2020/180770,
any SOS1 inhibitor example 1 to 540 as disclosed in WO 2020/180770,
SOS1 inhibitor generically described and/or specifically disclosed in WO 2018/172250,
any SOS1 inhibitor example 1 to 458 as disclosed in WO 2018/172250,
SOS1 inhibitor generically described and/or specifically disclosed in WO 2019/201848, and
any SOS1 inhibitor example 1 to 100 as disclosed in WO 2019/201848,

the disclosure being incorporated by reference in its entirety herein, and with the respective synthesis and properties.

Furthermore, referring to all aspects and embodiments, including methods of treatment, uses, combinations, and compositions, the SOS1 inhibitor can be selected from the group consisting of:
SOS1 inhibitor generically described and/or specifically disclosed in WO 2021/074227,
any SOS1 inhibitor example 1 to 342 as disclosed in WO 2021/074227,
SOS1 inhibitor generically described and/or specifically disclosed in WO 2021/092115,
any SOS1 inhibitor examples 1 to 71 and P (table A, tables 1 to 21) as disclosed in WO 2021/092115,
SOS1 inhibitor generically described and/or specifically disclosed in WO 2021/130731,
any SOS1 inhibitor example/compound 1 to 152 as disclosed in WO 2021/130731,
SOS1 inhibitor generically described and/or specifically disclosed in WO CN113200981,
any SOS1 inhibitor example/compound 1 to 22 as disclosed in CN113200981,
SOS1 inhibitor generically described and/or specifically disclosed in WO 2021/249519,
any SOS1 inhibitor example/compound 1 to 29 as disclosed in WO 2021/249519,
SOS1 inhibitor generically described and/or specifically disclosed in WO 2021/249475,
any SOS1 inhibitor examples 1 to 112 as disclosed in WO 2021/249475,
SOS1 inhibitor generically described and/or specifically disclosed in WO 2021/173524,
SOS1 inhibitor generically described and/or specifically disclosed in WO 2021/127429,
SOS1 inhibitor generically described and/or specifically disclosed in WO 2022/026465,
SOS1 inhibitor generically described and/or specifically disclosed in WO 2021/203768,
SOS1 inhibitor generically described and/or specifically disclosed in WO 2021/228028,
SOS1 inhibitor generically described and/or specifically disclosed in WO 2022/017339,
SOS1 inhibitor generically described and/or specifically disclosed in WO 2022/017519,
SOS1 inhibitor generically described and/or specifically disclosed in WO 2022/026465,
SOS1 inhibitor generically described and/or specifically disclosed in WO 2022/028506,
SOS1 inhibitor generically described and/or specifically disclosed in WO 2022/058344,
SOS1 inhibitor generically described and/or specifically disclosed in CN113801114,

the disclosure being incorporated by reference in its entirety herein, and with the respective synthesis and properties.

The term “SOS1 inhibitor” as used herein also includes the SOS1 inhibitors listed above in the form of a tautomer, of a pharmaceutically acceptable salt, of a hydrate or of a solvate, including a hydrate or solvate of a pharmaceutically acceptable salt. It also includes the SOS1 inhibitor in all its solid, preferably crystalline, forms and in all the crystalline forms of its pharmaceutically acceptable salts, hydrates and solvates, including hydrates and solvates of pharmaceutically acceptable salts.

The term “pharmaceutically acceptable salts” as used herein includes both acid and base addition salts. Pharmaceutically acceptable acid addition salts refers to those salts which retain the biological effectiveness and properties of the free bases and which are not biologically or otherwise undesirable, formed with inorganic acids or organic acids. Pharmaceutically acceptable base addition salts include salts derived from inorganic bases or organic nontoxic bases. The term “solvate” as used herein refers to an association or complex of one or more solvent molecules and a compound of the present invention. Examples of solvents include water, isopropanol, ethanol, methanol, DMSO, ethyl acetate, acetic acid and ethanolamine. The term “hydrate” refers to a complex where the solvent molecule is water.

In one embodiment the SOS1 inhibitor is compound I-1 or a pharmaceutically acceptable salt thereof. In another embodiment the SOS1 inhibitor is compound I-2 or a pharmaceutically acceptable salt thereof. In another embodiment the SOS1 inhibitor is compound I-3 or a pharmaceutically acceptable salt thereof. In another embodiment the SOS1 inhibitor is compound I-4 or a pharmaceutically acceptable salt thereof. In another embodiment the SOS1 inhibitor is compound I-13 or a pharmaceutically acceptable salt thereof. In another embodiment the SOS1 inhibitor is compound I-20 or a pharmaceutically acceptable salt thereof. In another embodiment the SOS1 inhibitor is compound I-21 or a pharmaceutically acceptable salt thereof. In another embodiment the SOS1 inhibitor is compound I-22 or a pharmaceutically acceptable salt thereof. In another embodiment the SOS1 inhibitor is compound I-23 or a pharmaceutically acceptable salt thereof. In another embodiment the SOS1 inhibitor is compound I-25 or a pharmaceutically acceptable salt thereof. In another embodiment the SOS1 inhibitor is compound I-26 or a pharmaceutically acceptable salt thereof. In another embodiment the SOS1 inhibitor is compound I-37 or a pharmaceutically acceptable salt thereof. In another embodiment the SOS1 inhibitor is compound I-38 or a pharmaceutically acceptable salt thereof. In another embodiment the SOS1 inhibitor is compound I-45 or a pharmaceutically acceptable salt thereof. In another embodiment the SOS1 inhibitor is compound I-49 or a pharmaceutically acceptable salt thereof. In another embodiment the SOS1 inhibitor is compound I-50 or a pharmaceutically acceptable salt thereof. In another embodiment the SOS1 inhibitor is compound I-52 or a pharmaceutically acceptable salt thereof. In another embodiment the SOS1 inhibitor is compound I-53 or a pharmaceutically acceptable salt thereof. In another embodiment the SOS1 inhibitor is compound I-54 or a pharmaceutically acceptable salt thereof. In another embodiment the SOS1 inhibitor is compound I-55 or a pharmaceutically acceptable salt thereof. In another embodiment the SOS1 inhibitor is compound I-57 or a pharmaceutically acceptable salt thereof. In another embodiment the SOS1 inhibitor is compound I-58 or a pharmaceutically acceptable salt thereof. In another embodiment the SOS1 inhibitor is compound I-59 or a pharmaceutically acceptable salt thereof. In another embodiment the SOS1 inhibitor is compound I-61 or a pharmaceutically acceptable salt thereof. In another embodiment the SOS1 inhibitor is compound I-69 or a pharmaceutically acceptable salt thereof. In another embodiment the SOS1 inhibitor is compound I-71 or a pharmaceutically acceptable salt thereof. In another embodiment the SOS1 inhibitor is compound I-73 or a pharmaceutically acceptable salt thereof. In another embodiment the SOS1 inhibitor is compound I-77 or a pharmaceutically acceptable salt thereof. In another embodiment the SOS1 inhibitor is compound I-78 or a pharmaceutically acceptable salt thereof. In another embodiment the SOS1 inhibitor is compound I-82 or a pharmaceutically acceptable salt thereof. In another embodiment the SOS1 inhibitor is compound I-87 or a pharmaceutically acceptable salt thereof. In another embodiment the SOS1 inhibitor is compound I-96 or a pharmaceutically acceptable salt thereof. In another embodiment the SOS1 inhibitor is compound I-97 or a pharmaceutically acceptable salt thereof. In another embodiment the SOS1 inhibitor is compound I-98 or a pharmaceutically acceptable salt thereof. In another embodiment the SOS1 inhibitor is compound I-99 or a pharmaceutically acceptable salt thereof. In another embodiment the SOS1 inhibitor is compound I-100 or a pharmaceutically acceptable salt thereof. In another embodiment the SOS1 inhibitor is compound I-101 or a pharmaceutically acceptable salt thereof. In another embodiment the SOS1 inhibitor is compound I-102 or a pharmaceutically acceptable salt thereof. In another embodiment the SOS1 inhibitor is compound I-103 or a pharmaceutically acceptable salt thereof. In another embodiment the SOS1 inhibitor is compound I-121 or a pharmaceutically acceptable salt thereof. In another embodiment the SOS1 inhibitor is compound I-123 or a pharmaceutically acceptable salt thereof. In another embodiment the SOS1 inhibitor is compound I-126 or a pharmaceutically acceptable salt thereof. In another embodiment the SOS1 inhibitor is compound I-128 or a pharmaceutically acceptable salt thereof. In another embodiment the SOS1 inhibitor is compound I-130 or a pharmaceutically acceptable salt thereof. In another embodiment the SOS1 inhibitor is compound I-156 or a pharmaceutically acceptable salt thereof. In another embodiment the SOS1 inhibitor is compound I-157 or a pharmaceutically acceptable salt thereof. In another embodiment the SOS1 inhibitor is compound BI-3406 or a pharmaceutically acceptable salt thereof. All these embodiments are preferred embodiments in respect of the nature of the SOS1 inhibitor.

### MEK Inhibitor

As used herein, the term “MEK” refers to the mitogen-activated protein kinase kinase. As used herein, the term “MEK inhibitor” refers to a compound that inhibits and/or reduces the biological activity of MEK. MEK inhibitors belonging to different compound classes are known.

Preferably, referring to all aspects and embodiments, including methods of treatment, uses, combinations, and compositions, the MEK inhibitor is selected from the group consisting of:
MEK inhibitors generically described and/or specifically disclosed in WO 2013/136249,
any MEK inhibitor example 1 to 79 as disclosed in WO 2013/136249
MEK inhibitors generically described and/or specifically disclosed in WO 2013/136254,
any MEK inhibitor example 1 to 21 as disclosed in WO 2013/136254,
MEK inhibitors generically described and/or specifically disclosed in WO 2005/121142, and
any MEK inhibitor example compound [I] disclosed in WO 2005/121142,

the disclosure being incorporated by reference in its entirety herein, and with the respective synthesis and properties.

The term “MEK inhibitor” as used herein also includes the MEK inhibitors listed above in the form of a tautomer, of a pharmaceutically acceptable salt, of a hydrate or of a solvate, including a hydrate or solvate of a pharmaceutically acceptable salt. It also includes the MEK inhibitor in all its solid, preferably crystalline, forms and in all the crystalline forms of its pharmaceutically acceptable salts, hydrates and solvates, including hydrates and solvates of pharmaceutically acceptable salts.

The MEK inhibitor may also be selected from the group consisting of trametinib, cobimetinib, binimetinib, selumetinib, refametinib and pharmaceutically acceptable salts thereof.

The compound denoted trametinib according to INN is a small-molecule inhibitor of MEK according to formula (T) or a pharmaceutically acceptable salt thereof or a hydrate or solvate, e.g. DMSO solvate, thereof

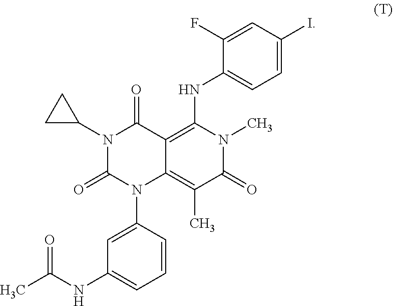 [SMILES: CC(=O)Nc1cccc(-n2c(=O)n(C3CC3)c(=O)c3c(Nc4ccc(I)cc4F)n(C)c(=O)c(C)c32)c1]

WO 2005/121142 describes trametinib as example 4-1. The compound is commercially available.

Also the compounds denoted under their INN names cobimetinib (ATC code: L01XE38), binimetinib (ATC code: L01XE41), selumetinib (ATC code: L01EE04), refametinib (BAY 869766) are known in the art and/or are commercially available.

More preferably, the MEK inhibitor is selected from the group consisting of the following specific MEK inhibitors or salts thereof: 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, 1-72, 1-73, 1-74, 1-75, 1-76, 1-77, 1-78, 1-79, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, and 2-21. All the listed MEK inhibitors are disclosed in WO 2013/136249 and WO 2013/136254, with the respective synthesis and properties.

In one embodiment the MEK inhibitor is trametinib or a pharmaceutically acceptable salt thereof. In another embodiment the MEK inhibitor is cobimetinib or a pharmaceutically acceptable salt thereof. In another embodiment the MEK inhibitor is binimetinib or a pharmaceutically acceptable salt thereof. In another embodiment the MEK inhibitor is selumetinib or a pharmaceutically acceptable salt thereof. In another embodiment the MEK inhibitor is refametinib or a pharmaceutically acceptable salt thereof. In one embodiment the MEK inhibitor is compound 1-1 or a pharmaceutically acceptable salt thereof. In one embodiment the MEK inhibitor is compound 1-2 or a pharmaceutically acceptable salt thereof. In one embodiment the MEK inhibitor is compound 1-3 or a pharmaceutically acceptable salt thereof. In one embodiment the MEK inhibitor is compound 1-4 or a pharmaceutically acceptable salt thereof. In another embodiment the MEK inhibitor is compound 1-5 or a pharmaceutically acceptable salt thereof. In one embodiment the MEK inhibitor is compound 1-6 or a pharmaceutically acceptable salt thereof. In one embodiment the MEK inhibitor is compound 1-7 or a pharmaceutically acceptable salt thereof. In one embodiment the MEK inhibitor is compound 1-8 or a pharmaceutically acceptable salt thereof. In another embodiment the MEK inhibitor is compound 1-9 or a pharmaceutically acceptable salt thereof. In one embodiment the MEK inhibitor is compound 1-10 or a pharmaceutically acceptable salt thereof. In one embodiment the MEK inhibitor is compound 1-11 or a pharmaceutically acceptable salt thereof. In one embodiment the MEK inhibitor is compound 1-12 or a pharmaceutically acceptable salt thereof. In one embodiment the MEK inhibitor is compound 1-13 or a pharmaceutically acceptable salt thereof. In one embodiment the MEK inhibitor is compound 1-14 or a pharmaceutically acceptable salt thereof. In one embodiment the MEK inhibitor is compound 1-15 or a pharmaceutically acceptable salt thereof. In another embodiment the MEK1 inhibitor is compound 1-16 or a pharmaceutically acceptable salt thereof. In one embodiment the MEK inhibitor is compound 1-17 or a pharmaceutically acceptable salt thereof. In one embodiment the MEK inhibitor is compound 1-18 or a pharmaceutically acceptable salt thereof. In one embodiment the MEK inhibitor is compound 1-19 or a pharmaceutically acceptable salt thereof. In one embodiment the MEK inhibitor is compound 1-20 or a pharmaceutically acceptable salt thereof. In one embodiment the MEK inhibitor is compound 1-21 or a pharmaceutically acceptable salt thereof. In one embodiment the MEK inhibitor is compound 1-22 or a pharmaceutically acceptable salt thereof. In one embodiment the MEK inhibitor is compound 1-23 or a pharmaceutically acceptable salt thereof. In one embodiment the MEK inhibitor is compound 1-24 or a pharmaceutically acceptable salt thereof. In one embodiment the MEK inhibitor is compound 1-25 or a pharmaceutically acceptable salt thereof. In one embodiment the MEK inhibitor is compound 1-26 or a pharmaceutically acceptable salt thereof. In one embodiment the MEK inhibitor is compound 1-27 or a pharmaceutically acceptable salt thereof. In one embodiment the MEK inhibitor is compound 1-28 or a pharmaceutically acceptable salt thereof. In another embodiment the MEK inhibitor is compound 1-29 or a pharmaceutically acceptable salt thereof. In one embodiment the MEK inhibitor is compound 1-30 or a pharmaceutically acceptable salt thereof. In one embodiment the MEK inhibitor is compound 1-31 or a pharmaceutically acceptable salt thereof. In one embodiment the MEK inhibitor is compound 1-32 or a pharmaceutically acceptable salt thereof. In one embodiment the MEK inhibitor is compound 1-33 or a pharmaceutically acceptable salt thereof. In one embodiment the MEK inhibitor is compound 1-34 or a pharmaceutically acceptable salt thereof. In another embodiment the MEK inhibitor is compound 1-35 or a pharmaceutically acceptable salt thereof. In one embodiment the MEK inhibitor is compound 1-36 or a pharmaceutically acceptable salt thereof. In another embodiment the MEK inhibitor is compound 1-37 or a pharmaceutically acceptable salt thereof. In one embodiment the MEK inhibitor is compound 1-38 or a pharmaceutically acceptable salt thereof. In one embodiment the MEK inhibitor is compound 1-39 or a pharmaceutically acceptable salt thereof. In one embodiment the MEK inhibitor is compound 1-40 or a pharmaceutically acceptable salt thereof. In one embodiment the MEK inhibitor is compound 1-41 or a pharmaceutically acceptable salt thereof. In one embodiment the MEK inhibitor is compound 1-42 or a pharmaceutically acceptable salt thereof. In one embodiment the MEK inhibitor is compound 1-43 or a pharmaceutically acceptable salt thereof. In one embodiment the MEK inhibitor is compound 1-44 or a pharmaceutically acceptable salt thereof. In one embodiment the MEK inhibitor is compound 1-45 or a pharmaceutically acceptable salt thereof. In one embodiment the MEK inhibitor is compound 1-46 or a pharmaceutically acceptable salt thereof. In one embodiment the MEK inhibitor is compound 1-47 or a pharmaceutically acceptable salt thereof. In one embodiment the MEK inhibitor is compound 1-48 or a pharmaceutically acceptable salt thereof. In one embodiment the MEK inhibitor is compound 1-49 or a pharmaceutically acceptable salt thereof. In one embodiment the MEK inhibitor is compound 1-50 or a pharmaceutically acceptable salt thereof. In one embodiment the MEK inhibitor is compound 1-51 or a pharmaceutically acceptable salt thereof. In one embodiment the MEK inhibitor is compound 1-52 or a pharmaceutically acceptable salt thereof. In one embodiment the MEK inhibitor is compound 1-53 or a pharmaceutically acceptable salt thereof. In one embodiment the MEK inhibitor is compound 1-54 or a pharmaceutically acceptable salt thereof. In one embodiment the MEK inhibitor is compound 1-55 or a pharmaceutically acceptable salt thereof. In one embodiment the MEK inhibitor is compound 1-56 or a pharmaceutically acceptable salt thereof. In another embodiment the MEK inhibitor is compound 1-57 or a pharmaceutically acceptable salt thereof. In one embodiment the MEK inhibitor is compound 1-58 or a pharmaceutically acceptable salt thereof. In one embodiment the MEK inhibitor is compound 1-59 or a pharmaceutically acceptable salt thereof. In one embodiment the MEK inhibitor is compound 1-60 or a pharmaceutically acceptable salt thereof. In one embodiment the MEK inhibitor is compound 1-61 or a pharmaceutically acceptable salt thereof. In one embodiment the MEK inhibitor is compound 1-62 or a pharmaceutically acceptable salt thereof. In one embodiment the MEK inhibitor is compound 1-63 or a pharmaceutically acceptable salt thereof. In one embodiment the MEK inhibitor is compound 1-64 or a pharmaceutically acceptable salt thereof. In one embodiment the MEK inhibitor is compound 1-65 or a pharmaceutically acceptable salt thereof. In one embodiment the MEK inhibitor is compound 1-66 or a pharmaceutically acceptable salt thereof. In one embodiment the MEK inhibitor is compound 1-67 or a pharmaceutically acceptable salt thereof. In one embodiment the MEK inhibitor is compound 1-68 or a pharmaceutically acceptable salt thereof. In one embodiment the MEK inhibitor is compound 1-69 or a pharmaceutically acceptable salt thereof. In one embodiment the MEK inhibitor is compound 1-70 or a pharmaceutically acceptable salt thereof. In one embodiment the MEK inhibitor is compound 1-71 or a pharmaceutically acceptable salt thereof. In one embodiment the MEK inhibitor is compound 1-72 or a pharmaceutically acceptable salt thereof. In one embodiment the MEK inhibitor is compound 1-73 or a pharmaceutically acceptable salt thereof. In one embodiment the MEK inhibitor is compound 1-74 or a pharmaceutically acceptable salt thereof. In one embodiment the MEK inhibitor is compound 1-75 or a pharmaceutically acceptable salt thereof. In one embodiment the MEK inhibitor is compound 1-76 or a pharmaceutically acceptable salt thereof. In another embodiment the MEK inhibitor is compound 1-77 or a pharmaceutically acceptable salt thereof. In another embodiment the MEK inhibitor is compound 1-78 or a pharmaceutically acceptable salt thereof. In another embodiment the MEK inhibitor is compound 1-79 or a pharmaceutically acceptable salt thereof. In another embodiment the MEK inhibitor is compound 2-1 or a pharmaceutically acceptable salt thereof. In another embodiment the MEK inhibitor is compound 2-2 or a pharmaceutically acceptable salt thereof. In another embodiment the MEK inhibitor is compound 2-3 or a pharmaceutically acceptable salt thereof. In another embodiment the MEK inhibitor is compound 2-4 or a pharmaceutically acceptable salt thereof. In another embodiment the MEK inhibitor is compound 2-5 or a pharmaceutically acceptable salt thereof. In another embodiment the MEK inhibitor is compound 2-6 or a pharmaceutically acceptable salt thereof. In another embodiment the MEK inhibitor is compound 2-7 or a pharmaceutically acceptable salt thereof. In another embodiment the MEK inhibitor is compound 2-8 or a pharmaceutically acceptable salt thereof. In another embodiment the MEK inhibitor is compound 2-9 or a pharmaceutically acceptable salt thereof. In another embodiment the MEK inhibitor is compound 2-10 or a pharmaceutically acceptable salt thereof. In another embodiment the MEK inhibitor is compound 2-11 or a pharmaceutically acceptable salt thereof. In another embodiment the MEK inhibitor is compound 2-12 or a pharmaceutically acceptable salt thereof. In another embodiment the MEK inhibitor is compound 2-13 or a pharmaceutically acceptable salt thereof. In another embodiment the MEK inhibitor is compound 2-14 or a pharmaceutically acceptable salt thereof. In another embodiment the MEK inhibitor is compound 2-15 or a pharmaceutically acceptable salt thereof. In another embodiment the MEK inhibitor is compound 2-16 or a pharmaceutically acceptable salt thereof. In another embodiment the MEK inhibitor is compound 2-17 or a pharmaceutically acceptable salt thereof. In another embodiment the MEK inhibitor is compound 2-18 or a pharmaceutically acceptable salt thereof. In another embodiment the MEK inhibitor is compound 2-19 or a pharmaceutically acceptable salt thereof. In another embodiment the MEK inhibitor is compound 2-20 or a pharmaceutically acceptable salt thereof. In another embodiment the MEK inhibitor is compound 2-21 or a pharmaceutically acceptable salt thereof. All these embodiments are preferred embodiments in respect of the nature of the MEK inhibitor.

### Combinations

The combination of all embodiments in respect of the nature of the SOS1 inhibitor as described herein with all embodiments in respect of the nature of the MEK inhibitor as described herein results in specific combinations or groups of combinations which shall all be deemed to be specifically disclosed and to be embodiments of the invention and of all of its combinations, compositions, kits, methods, uses and compounds for use. Preferred embodiments are combinations of embodiments I-1, I-2, I-3, I-4, I-13, I-20, I-21, I-22, I-23, I-25, I-26, I-37, I-38, I-45, I-49, I-50, I-52, I-53, I-54, I-55, I-57, I-58, I-59, I-61, I-69, I-71, I-73, I-77, I-78, I-82, I-87, I-96, I-97, I-98, I-99, I-100, I-101, I-102, I-103, I-121, I-123, I-126, I-128, I-130, I-156, I-157 and BI-3406 or a pharmaceutically acceptable salt thereof in respect of the nature of the SOS1 inhibitor with embodiments trametinib, cobimetinib, binimetinib, selumetinib, refametinib, 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 1-40, 1-41, 1-42, 1-43, 1-44, 1-45, 1-46, 1-47, 1-48, 1-49, 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, 1-72, 1-73, 1-74, 1-75, 1-76, 1-77, 1-78, 1-79, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, and 2-21 or a pharmaceutically acceptable salt thereof in respect of the nature of the MEK inhibitor.

To be used for treatment, the SOS1 inhibitor and the MEK inhibitor, separately or jointly, may be included into pharmaceutical compositions appropriate to facilitate administration. The compounds thus may be formulated, alone or together, in suitable dosage unit formulations containing conventional non-toxic pharmaceutically acceptable carriers, excipients and/or vehicles appropriate for each route of administration. Typical pharmaceutical compositions for administering the SOS1 inhibitor and the MEK inhibitor, separately or jointly, include for example tablets, capsules, suppositories, solutions, e.g. solutions for injection and infusion, elixirs, emulsions or dispersible powders. Dosage forms and formulations of active ingredients are known in the art.

The SOS1 inhibitor may be administered by oral routes of administration and may be formulated, alone or together, in suitable dosage unit formulations containing conventional non-toxic pharmaceutically acceptable carriers, excipients and vehicles appropriate for each route of administration. Likewise, the MEK inhibitor may be administered by oral routes of administration and may be formulated, alone or in combination, in suitable dosage unit formulations containing conventional non-toxic pharmaceutically acceptable carriers, excipients and vehicles appropriate for each route of administration. Although oral administration may be preferred in view of compliance, routes of administration for the SOS1 inhibitor and/or MEK inhibitor described herein, are not limited to oral administration, but the compounds may be administered parenterally, e.g. intramuscular, intraperitoneal, intravenous, transdermal or subcutaneous injection or by implant, or enterical, nasal, vaginal, rectal, or topical administration.

### Single Therapy and Combination Therapy

It is a purpose of the present invention to provide secondary treatment option for cancers with acquired resistance to KRAS G12C inhibitors.

It was discovered in vitro in cell line models that clones with secondary Y96D or Y96S mutations were resistant to both KRAS G12C inhibitors sotorasib and adagrasib. Surprisingly, a SOS1 inhibitor, such as BI-3406, and a combination of the SOS1 inhibitor and a MEK inhibitor, such as trametinib, showed potent activity against cells exhibiting a primary G12C mutation plus a secondary Y96D and/or Y96S (Y96D/S) mutation.

Specifically, treatment with SOS1 inhibitor BI-3406 and combination treatment with SOS1 inhibitor BI-3406 and MEK inhibitor trametinib reduced cell proliferation.

Although cancer treatment with KRAS G12C inhibitors, such as sotorasib and adagrasib, is known in the art, therapeutic concepts for a secondary treatment option for cancers with acquired resistance to KRAS G12C inhibitors are still lacking.

Thus, the invention relates to SOS1 inhibitors, MEK inhibitors, or combinations of SOS1 inhibitors with MEK inhibitors, as described herein, for use in anti-cancer therapy when the cancer is resistant to treatment with an inhibitor of KRAS G12C. For single treatment, the SOS1 inhibitor or the MEK inhibitor can be administered formulated in a pharmaceutical composition or dosage form. A combined treatment may include that the SOS1 inhibitor and the MEK inhibitor can be administered formulated either dependently, such as formulated together into one composition, or independently, such as formulated as separate compositions. In other words, in combination therapy, the SOS1 inhibitor and the MEK inhibitor may be administered either as part of the same pharmaceutical composition or dosage form or, preferably, in separate pharmaceutical compositions or dosage forms.

Another aspect relates to a pharmaceutical composition comprising as active ingredient a SOS1 inhibitor as described herein. A further aspect relates to a pharmaceutical composition comprising as active ingredient a MEK inhibitor as described herein. Another aspect relates to a pharmaceutical combination comprising as active ingredients a SOS1 inhibitor and a MEK inhibitor both as described herein and optionally pharmaceutically acceptable carrier, excipients, and/or vehicles, for use in the treatment and/or prevention of cancer, wherein the cancer is resistant to treatment with an inhibitor of KRAS G12C.

The term “pharmaceutically acceptable carrier, excipients and/or vehicles” refers to a non-toxic carrier, excipient or vehicle that does not destroy the pharmacological activity of the compound with which it is formulated. Pharmaceutically acceptable carriers, excipients or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, polyvinyl pyrrolidone, cellulose-based substances, sodium carboxymethylcellulose, or polyethylene glycol.

As used herein, the term “active ingredient” refers to a component that is intended to furnish pharmacological activity or other direct effect.

The SOS1 inhibitor, or the MEK inhibitor, or the combination of a SOS1 inhibitor and a MEK inhibitor may be administered at therapeutically effective amounts or be included in a pharmaceutical composition, dosage form or pharmaceutical combination in a therapeutically effective amount. A “therapeutically effective amount” refers to an amount effective at dosages and for periods of time necessary to achieve a desired therapeutic result and is the minimum amount necessary to prevent, ameliorate, or treat a disease or disorder, or which any toxic or detrimental effects of the compound is outweighed by the therapeutically beneficial effects.

As used herein, the term “pharmaceutical combination” may refer to either a fixed combination in one pharmaceutical composition or dosage unit form, or, preferably, a kit of parts for the combined administration where the SOS1 inhibitor may be administered independently of the MEK inhibitor at the same time or separately within time intervals. The compounds of the pharmaceutical combination can be together or separate. This means that the pharmaceutical combination of SOS1 inhibitor and a MEK inhibitor refers to use, application or formulations of the separate partners with or without instructions for combined use or to combination products. The combination partners may thus be administered entirely separately or be entirely separate pharmaceutical dosage forms. The combination partners may be pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active. The terms “co-administration” or “combined administration” or “combined use” or the like as utilized herein are meant to encompass administration of the selected combination partner to a single subject in need thereof (e.g. a patient), and are intended to include treatment regimens in which the active ingredients are not necessarily administered by the same route of administration and/or at the same time.

Another aspect relates to a kit for use in the treatment and/or prevention of cancer, wherein the cancer is resistant to treatment with an inhibitor of KRAS G12C, the kit comprising in one or more containers:
(i) a (first) pharmaceutical composition or dosage form comprising a SOS1 inhibitor as described herein, or a pharmaceutically acceptable salt thereof, and, optionally, pharmaceutically acceptable carriers, excipients and/or vehicles; and/or
(ii) a (second) pharmaceutical composition or dosage form comprising a MEK as described herein, and, optionally, pharmaceutically acceptable carriers, excipients and/or vehicles; and
(iii) optionally a package insert comprising instructions.

The terms “first” and “second” with respect to pharmaceutical compositions, as used herein, is solely intended to indicate that these compositions are two different compositions. Thus, these terms shall not be understood to refer to the order or sequence of administration.

Preferably, the package insert comprises printed instructions for simultaneous, concurrent, sequential, successive, alternate or separate use in the treatment of a hyperproliferative disease, in particular cancer, as described herein, in a patient in need thereof.

The SOS1 inhibitor, the MEK inhibitor, and the combination of SOS1 inhibitor and MEK inhibitor, the pharmaceutical compositions and combination, as well as all formulations of SOS1 inhibitor and the MEK inhibitor as disclosed herein, can be administered simultaneously, concurrently, sequentially, successively, alternately or separately.

The term “simultaneous” refers to the administration of both compounds/compositions at substantially the same time. The term “concurrent” refers to administration of the active ingredients within the same general time period, for example on the same day(s) but not necessarily at the same time. The term “sequential” administration includes administration of one active ingredient during a first time period, for example over the course of a few hours, days or a week, using one or more doses, followed by administration of the other active ingredient during a second time period, for example over the course of a few hours, days or a week, using one or more doses. An overlapping schedule may also be employed, which includes administration of the active ingredients on different days over the treatment period, not necessarily according to a regular sequence. The term “successive” administration, alternatively, refers to an administration where the second administration step is carried out immediately once the administration of the first compounds has been finished. Alternate administration includes administration of one active ingredient during a time period, for example over the course of a few hours, days or a week, followed by administration of the other active ingredient during a subsequent period of time, for example over the course of a few hours, days or a week, and then repeating the pattern for one or more cycles, wherein the overall number of repeats depends on the chosen dosage regimen. Variations of these general administration forms may also be employed.

### KRAS G12C Inhibitors

The term “KRAS G12C inhibitor” as used herein refers to a compound that inhibits and/or reduces the biological activity of KRAS exhibiting a G12C mutation. KRAS G12C inhibitors belonging to different compound classes are known.

Examples are sotorasib (AMG 510) and adagrasib (MRTX 849). Sotorasib and adagrasib are KRAS G12C selective inhibitors for which clinical data have been reported. The compound known under the INN adagrasib is also known under lab code MRTX 849. For example, WO 2017/201161 and WO 2019/099524 describe general reaction schemes for preparation, synthetic routes, and the properties. The compound known under the INN sotorasib is also known under lab code AMG 510. For example, WO 2018/217651 and WO 2020/102730 describe general reaction schemes for preparation, synthetic routes, and the properties. The term “KRAS G12C inhibitor” as used herein also encompasses the tautomers and pharmaceutically acceptable salts and all other solid forms of the compound.

Further examples are compounds known as JNJ-74699157 and LY3499446.

JNJ-74699157 (ARS-3248) is another small molecule KRAS G12C inhibitor that recently reached clinical testing in humans. Also the KRAS G12C inhibitor known as LY3499446 is known to enter phase 1 studies (Nagasaka et al., Cancer Treat Rev., 2020, 84, 101974).

### Cancers

The combinations, compositions, kits, uses, methods and compounds for use according to the present invention are usable for the treatment of cancer that is resistant to treatment with an inhibitor of KRAS G12C.

According to an aspect is provided a SOS1 inhibitor and/or a MEK inhibitor for use in the treatment and/or prevention of cancer, wherein the SOS1 inhibitor or the MEK inhibitor is administered alone or the SOS1 inhibitor is administered in combination with the MEK inhibitor, where the cancer is resistant to treatment with an inhibitor of KRAS G12C.

Another aspect refers to the aforementioned pharmaceutical compositions or combination or kit for use in a method of treating and/or preventing of cancer, wherein the cancer is resistant to treatment with an inhibitor of KRAS G12C as described herein.

Another aspect relates to a method of treating and/or preventing cancer, the method comprising administering to a patient a therapeutically effective amount of a SOS1 inhibitor, or a therapeutically effective amount of a MEK inhibitor, or a therapeutically effective amount of a SOS1 inhibitor in combination with a therapeutically effective amount of a MEK inhibitor, wherein the cancer is resistant to treatment with an inhibitor of KRAS G12C.

Another aspect relates to a use of a SOS1 inhibitor and/or a MEK inhibitor for the manufacture of a medicament for use in the treatment and/or prevention of cancer, wherein the SOS1 inhibitor or the MEK inhibitor is to be used alone or the SOS1 inhibitor is to be used in combination with the MEK inhibitor, wherein the cancer is resistant to treatment with an inhibitor of KRAS G12C.

Patients with relapse and/or with resistance to one or more KRAS G12C inhibitor are particularly amenable for combined treatment according to this invention, such as for second or third line treatment cycles (optionally in further combination with one or more other anti-cancer agents), or as add-on combination or as replacement treatment.

The therapeutic applicability of the SOS1 inhibitor or the MEK inhibitor or the combination of a SOS1 inhibitor and a MEK inhibitor thus may include second line, third line or further lines of treatment of patients. The cancer may be recurrent, relapsed, resistant or refractory to one or more anti-cancer treatments using KRAS G12C inhibitors. Thus, the patients may have received previous anti-cancer therapies with one or more KRAS G12C inhibitor, which have not completely cured the disease.

Therapy using a SOS1 inhibitor or a MEK inhibitor or combination therapy using a SOS1 inhibitor and a MEK inhibitor may be effective at treating subjects whose cancer has relapsed, or whose cancer has become drug resistant to KRAS G12C inhibitors, or whose cancer has failed one, two or more lines of mono- or combination therapy with one or more KRAS G12C inhibitors. A cancer which initially responded to a KRAS G12C inhibitor can relapse and become resistant to the KRAS G12C inhibitor when the KRAS G12C inhibitor is no longer effective in treating the subject with the cancer, for example despite the administration of increased dosages of the KRAS G12C inhibitor.

Accordingly, in an embodiment of the combination, compositions, kits, uses, methods and compounds for use according to the invention, the cancer has become resistant to treatment with an inhibitor of KRAS G12C after earlier treatment with an inhibitor of KRAS G12C. In another embodiment, the cancer is resistant to treatment with sotorasib (AMG 510) and/or adagrasib (MRTX 849). In preferred embodiments, the cancer has become resistant to treatment with sotorasib (AMG 510) and/or adagrasib (MRTX 849) after earlier treatment with sotorasib (AMG 510) and/or adagrasib (MRTX 849).

As used herein, the term “cancer” refers to a malignant disease condition wherein cell growth is increased over normal levels. Cancers may be classified in several ways: by the type of tissue in which the cancer originates (histological type) and by primary site, or the location in the body, where the cancer first developed, and/or by exhibiting a molecular feature.

In a preferred embodiment of the combination, compositions, kits, uses, methods and compounds for use according to the invention, the cancer is defined as exhibiting a molecular feature, where the cancer cells of the cancer exhibit a primary KRAS G12C mutation and further exhibit a secondary mutation Y96X, preferably selected from the group consisting of Y96D and Y96S. In a preferred embodiment, the cancer cells of the cancer have been determined to exhibit a primary KRAS G12C mutation and to further exhibit a secondary mutation Y96X, preferably selected from the group consisting of Y96D and Y96S.

The combinations, compositions, kits, uses, methods and compounds for use according to the invention including all embodiments may be useful in the treatment of a variety of cancers, for example a cancer selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

Cancers of the lung include non-small cell lung cancer (NSCLC) and small cell lung cancer (SCLC). In an embodiment, the cancer is non-small cell lung cancer (NSCLC). In a further embodiment, the cancer is colon cancer. In a further embodiment, the cancer is pancreatic cancer.

KRAS G12C, Y96X and Y96D and/or Y96S Mutation

As used herein, the term “Y96X” refers to any substitution of tyrosine with another amino acid. Preferred are substitutions of tyrosine with serine (Y96S) or with aspartate (Y96D).

Determining whether a tumor or cancer comprises a KRAS G12C, Y96X, and preferably a Y96S and/or Y96D mutation can be undertaken by assessing the nucleotide sequence encoding the KRAS protein, by assessing the amino acid sequence of the KRAS protein, or by assessing the characteristics of a putative KRAS mutant protein. The sequence of wild-type human KRAS is known in the art. Methods for detecting a mutation in a KRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS mutations by real-time PCR. In real-time PCR, fluorescent probes specific for the KRAS mutation are used. When a mutation is present, the probe binds and fluorescence is detected. In some embodiments, the KRAS mutation is identified using a direct sequencing method of specific regions (e.g. exon 2 and/or exon 3) in the KRAS gene. This technique will identify all possible mutations in the region sequenced. Methods for detecting a mutation in a KRAS are known by those of skill in the art. These methods include, but are not limited to, detection of a KRAS mutant using a binding agent (e.g. an antibody) specific for the mutant protein, protein electrophoresis, Western blotting and direct peptide sequencing.

Methods for determining whether a tumor or cancer comprises a KRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA. In some embodiments the sample is a liquid biopsy and the test is done on a sample of blood to look for cancer cells from a tumor that are circulating in the blood or for pieces of DNA from tumor cells that are in the blood.

Another aspect is based on identifying a link between the KRAS mutation status of a patient and potential susceptibility to a treatment according to the invention. Treatment may be single treatment with either a SOS1 inhibitor or a MEK inhibitor or combination treatment with a SOS1 inhibitor and a MEK inhibitor. Such treatment may then advantageously be used to treat patients with KRAS mutations who may be resistant to other therapies. This therefore provides opportunities, methods and tools for selecting patients for treatment according to the invention, particularly cancer patients. The selection is based on whether the tumor cells to be treated are resistant or have become resistant to treatment with a KRAS G12C inhibitor, e.g. sotorasib (AMG 510) and/or adagrasib (MRTX 849), in particular if they exhibit G12C as a primary KRAS mutation and a secondary mutation Y96X, preferably selected from the group consisting of Y96D and Y96S. The KRAS gene status (G12C Y96X, preferably, G12C Y96D and/or G12C Y96S) could therefore be used as a biomarker to indicate that selecting treatment as described herein may be advantageous.

One aspect relates to an in vitro method for detecting or diagnosing that an individual has acquired resistance to treatment with an inhibitor of KRAS G12C and/or is susceptible to treatment with SOS1 inhibitor or a MEK inhibitor or a combination of a SOS1 inhibitor and a MEK inhibitor, the method comprising the step of:
determining, in a biological sample from an individual, the presence of a KRAS mutation Y96X, preferably selected from the group consisting of Y96D and Y96S, wherein the individual is classified as being resistant to treatment with an inhibitor of KRAS G12C and being susceptible to treatment with a SOS1 inhibitor or a MEK inhibitor or a combination of a SOS1 inhibitor and a MEK inhibitor, based on the detection of the KRAS mutation Y96X, preferably selected from the group consisting of Y96D and Y96S.

As used herein, the term “individual” refers to a test subject or patient.

According to another aspect, there is provided a method for selecting a patient for treatment with a SOS1 inhibitor or a MEK inhibitor or combination treatment with a SOS1 inhibitor and a MEK inhibitor, the method comprising
providing a tumor cell-containing sample from a patient;
determining whether the KRAS gene in the patient's tumor cell-containing sample exhibits a mutant KRAS with a primary mutation G12C and a secondary mutation Y96X, preferably selected from the group consisting of Y96D and Y96S (i.e. G12C Y96D or G12C Y96S, respectively); and

selecting a patient for treatment based thereon.

The method may include or exclude the actual patient sample isolation step.

The patient may be selected for treatment if the tumor cell DNA has a G12C Y96X mutant KRAS gene.

In one aspect, the patient is selected for treatment if the tumor cell DNA has a G12C Y96D mutant KRAS gene.

In another aspect, the patient is selected for treatment if the tumor cell DNA has a G12C Y96S mutant KRAS gene.

A further related aspect refers to the use of a KRAS mutation Y96X, preferably selected from the group consisting of Y96D and Y96S as a biomarker for a cancer or cancer cells being resistant to treatment with an inhibitor of KRAS G12C and/or the cancer or cancer cells being susceptible to treatment with a SOS1 inhibitor or a MEK inhibitor or a combination of a SOS1 inhibitor and a MEK inhibitor.

As used herein, the term “biomarker” refers to a biochemical parameter associated with the presence of a specific physiological state, such as resistancy to a KRAS G12C inhibitor. As described above, determination methods assessing the nucleotide sequence encoding the KRAS protein and/or methods for detecting the mutation in a KRAS nucleotide sequence can be used.

Unless otherwise defined, the technical and scientific terms used herein have the same meaning as commonly understood by one of ordinary skill in the art to which this invention belongs.

The examples which follow serve to illustrate the invention in more detail but do not constitute a limitation thereof.

### Cell Lines and Reagents:

NSCLC cell lines expressing KRAS G12 mutations (NCI-H358 (G12C), NCI-H23 (G12C), NCI-H2122 (G12C), NCI-H2009 (G12A) and NCI-H441 (G12V) cells) were kindly provided by late Dr. Adi F. Gazdar. A549 (G12S) and SK-LU1 (G12D) cells were a kind gift by late Dr. Hirotaka Osada. The immortalized murine pro-B cell line Ba/F3 was obtained from the RIKEN Bio Resource Center (Tsukuba, Japan).

Cells except for SK-LU1 cells were cultured in RPMI 1640 medium (Wako, Osaka, Japan) supplemented with 10% fetal bovine serum (FBS, Sigma-Aldrich, St. Louis, MO, USA) supplemented with 1% penicillin/streptomycin (P/S, Wako) at 37° C. with 5% CO2. SK-LU1 cells were cultured in DMEM (Sigma-Aldrich) with 10% FBS (Sigma-Aldrich) and 1% P/S (Wako). We maintained parental Ba/F3 cells in the presence of murine IL-3 under sterile conditions. Mycoplasma contamination was checked using the TaKaRa PCR Mycoplasma Detection Set (Takara, Kusatsu, Japan).

The KRAS G12C inhibitors sotorasib and adagrasib were purchased from MedChemExpress (Monmouth Junction, NJ, USA). BI-3406, a SOS1 inhibitor, was provided by Boehringer Ingelheim (Ingelheim, Germany). TNO155 was purchased from Selleck Chemicals (Houston, TX, USA). The drugs were dissolved in dimethyl sulfoxide (Sigma-Aldrich) at 10 mM and stored at −80° C.

### Methods

Introduction of KRAS Mutations into Ba/F3 and H358 Cells:

Introduction of G12C, G12D and G12V mutations into Ba/F3 cells or H358 cells was conducted using a retrovirus system as previously reported by Koga T. et al. (Lung Cancer 2018; 126:72-79.26) These three mutations account for ˜80% of KRAS mutations in NSCLC. Briefly, each KRAS mutation was introduced using a Prime STAR Mutagenesis Basal Kit (Takara) with designed primers into the pBABE-puro-KRas construct (Addgene, Cambridge, MA, USA). Retroviral particles were generated by co-transfection of each pBABE-puro-KRAS construct and pVSV-G vector (Clontech, Fremont, CA, USA) into gp-IRES 293 cells with FuGENE6 transfection reagent (Roche Diagnostics, Basel, Switzerland). Viral particles were concentrated using a retrovirus concentration kit (Clontech). Ba/F3 cells (3×103) or H358 cells (1×104) were transfected with each retrovirus and cultured at 37° C. for a few days. The transfected Ba/F3 cells or H358 cells were selected with 0.8-1.0 μg/mL or 2.0 μg/mL of puromycin, respectively. After puromycin selection, presence of each KRAS mutation was confirmed as follows; total RNA was extracted from cells using a mirVana miRNA Isolation Kit (Qiagen, Hilden, Germany), and cDNA was generated by reverse transcription using ReverTra Ace (TOYOBO, Osaka, Japan). The KRAS coding sequence was amplified by PCR. The KRAS nucleotide sequence was checked by Sanger sequencing using the 3130 or 3500XL Genetic Analyzer (Applied Biosystems, Waltham, MA).

### Cell Growth Assay and Growth Inhibition Assay:

Ba/F3 cells (3×104) expressing one of KRAS mutations were plated in six-well plates and cultured in the absence of IL-3. Non-transfected Ba/F3 cells were cultured with or without IL-3 as a control. The number of cells was counted in triplicate every 24 hours until 96 hours using a OneCell Counter (Biomedical Medical Science, Tokyo, Japan).

In the two-dimensional (2D) growth inhibition assay, 5x103 cells were cultured in 96-well plates for 24 hours and treated with reagents at ten different concentrations for 72 hr. Cell viability assays were performed using Cell Counting Kit-8 (Dojindo Laboratories, Kumamoto, Japan). The amount of formazan dye, which reflects cell viability, was measured by reading the absorbance at 450 nm using a multiplate reader (Tecan, Mannedorf, Switzerland). In the 3D growth inhibition assay, 5×103 cells were embedded in growth factor-reduced Matrigel (Corning, New York, USA) and cultured in RPMI 1640 medium (Wako) with 10% FBS (Sigma-Aldrich) and P/S (Wako) in 96-well plates. After 72 hours of incubation, cells were treated with the indicated concentrations for 72 hours, and a cell viability assay was performed as described above. IC50 values were determined by a nonlinear regression curve fit utilizing a variable slope model with normalized the response in GraphPad prism version 8 (GraphPad Software, San Diego, CA).

### Establishment of Clones Resistant to KRAS G12C Inhibitors:

ENU (Sigma-Aldrich) mutagenesis to generate clones resistant to sotorasib and adagrasib was performed as described (Kobayashi Y. et al., Mol Cancer Ther 2017; 16:357-364). A total of 1×106 Ba/F3 cells harboring KRAS G12C were exposed to 100 μg/ml ENU for 24 hours. After washing with PBS, cells were cultured for 48 hours and plated in 96-well plates with the indicated concentrations of KRASG12C inhibitors. The cells were treated for 14-21 days, and medium and drug were changed every 3rd to 5th day. Clones resistant to KRASG12C inhibitors were generated in the course of the treatment.

### Immunoblotting:

Preparation of the cell lysates and immunoblotting were conducted in a standard manner. After treatment with the indicated concentrations of KRASG12C inhibitors or DMSO, cell pellets were dissolved in lysis buffer. Protein concentration was measured using DC Protein Assay (Bio-Rad, Hercules, CA) with BSA as standard curve. A total of 20-30 μg protein was applied to each well of the 5-20% acrylamide and electrophoresed for 2.0 hr at 120V. Separated proteins were transferred to PVDF membranes (Trans-Blot® Turbo™ Mini 0.2 μm PVDF Transfer Pack, Bio-Rad) using a Trans-Blot® Turbo™ transfer system (Bio-Rad). After blocking with Blocking Buffer (TaKaRa), the membranes were probed with antibodies against the following proteins overnight at 4° C.: phospho (p)-p44/42 MAPK (Thr202/Tyr204, CST #9101s), p44/42 MAPK (CST #9102s), pMEK1 (S298, CST #9128s), MEK1 (CST #9146s), pS6 (S235/236, CST #4858s), S6 (CST #2217), KRAS (CST #53270s) and β-actin (CST #4970s). As a secondary antibody, HRP-conjugated anti-rabbit IgG (CST #7040) was incubated with the target protein and primary antibody complex for two hours. For chemiluminescence assays, ECL solution (GE Healthcare, Chicago, Illinois) was added to the membrane and scanned in an Amersham Imager 680 (GE Healthcare) to detect the expression of the target protein.

### Example 1

Determination of Secondary Mutations Developed Upon Treatment with Sotorasib or Adagrasib
1.1 Confirmation of Growth Inhibitory Effects of Sotorasib and Adagrasib in Cells with KRAS G12C Mutations

First, the growth inhibitory activity of the clinical-stage KRAS G12C inhibitors, sotorasib and adagrasib, was evaluated in six NSCLC cell lines carrying KRAS G12 mutations both in 2D and 3D culture as described above. Growth inhibition curves of KRAS G12 mutant NSCLC cell lines H358 (G12C), H23 (G12C), A549 (G12S), H2009 (G12A), H441 (G12V), and SK-LU1 (G12D) cells treated with sotorasib and adagrasib confirmed that both KRAS G12C inhibitors demonstrated growth inhibition activity in H358 and H2122 cells harboring the KRAS G12C mutation, while NSCLC cell lines with KRAS mutations other than G12C were resistant to sotorasib and adagrasib, with the exception of H23 cells that express KRAS G12C but had low sensitivity to sotorasib.

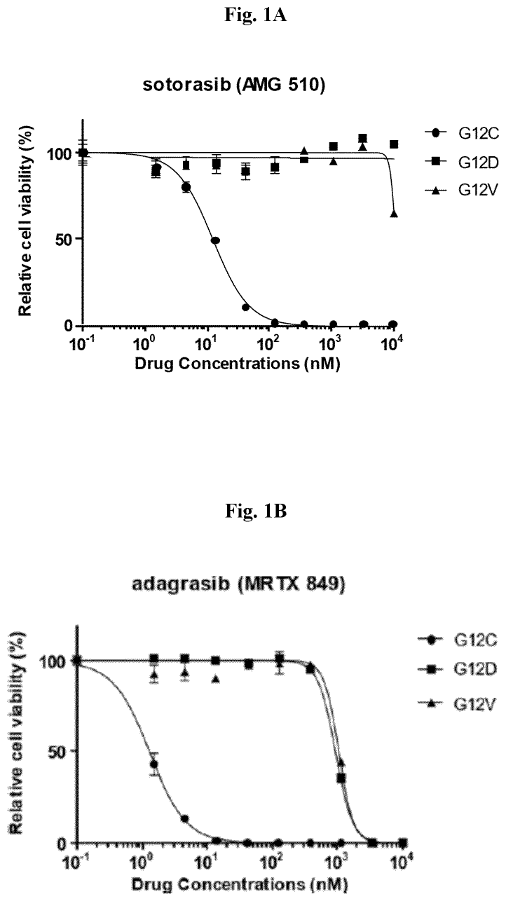

The growth inhibitory effects of sotorasib and adagrasib in Ba/F3 cell lines driven by KRAS G12 mutant variants KRAS G12C, G12V or G12D was also examined. The FIG. 1A shows the growth inhibition curves for engineered Ba/F3 cells harboring KRAS G12C, G12D or G12V treated with the indicated concentrations of sotorasib for 72 hours. The results are shown as the mean values of three individual experiments. Error bars indicate the standard deviation (SD). The FIG. 1B shows the growth inhibition curves for engineered Ba/F3 cells harboring KRAS G12C, G12D or G12V treated with the indicated concentrations of adagrasib for 72 hours. As can be seen from the FIGS. 1A and 1B, Ba/F3 cells expressing KRAS G12C were sensitive to sotorasib and adagrasib, while Ba/F3 cells with either KRAS G12D or G12V mutations were resistant. The IC50 values for sotorasib and adagrasib of Ba/F3 cells with KRAS G12C were 12.4 nM and 1.3 nM, respectively.

These results show that sotorasib or adagrasib are effective in H358 cells and in Ba/F3 cells exhibiting a KRAS G12C mutation.

1.2 Exploration for Secondary KRAS Mutations that Cause Resistance to Sotorasib or Adagrasib

To identify secondary mutations that would confer acquired resistance to sotorasib or adagrasib, ENU mutagenesis screening was conducted as described above. The minimum concentrations of sotorasib (100 nM) and adagrasib (20 nM) were determined as the lowest concentration that suppressed parental G12C Ba/F3 cell growth. The maximum concentrations of sotorasib (2000 nM) and adagrasib (1000 nM) were determined to exceed >100 times of IC50 of each KRASG12C inhibitor for G12C Ba/3 cells. In this experiment, a total of 142 resistant clones were generated. Among these clones, secondary KRAS mutations were identified in 124 of 142 clones corresponding to 87.3%. In the clones treated with sotorasib, 68 resistant clones carrying secondary KRAS mutations were identified. After treatment with high sotorasib concentrations (≥1000 nM), A59T (n=6), R68M (n=3) and Y96D (n=1) mutations were identified, while G13D (n=13), A59S (n=11), R68M (n=8) and Q61L (n=5) mutations were detected as most common in cells that grew in the lower concentrations of sotorasib. After adagrasib treatment, 74 resistant clones were derived. Y96D mutation was identified in cells established with high concentrations of adagrasib (≥500 nM; n=13), while Q99L (n=38), R68S (n=10), V8E (n=5), M72I (n=5) and A59S (n=3), were detected in cells that grew in the lower concentrations of adagrasib. Shared secondary mutations between sotorasib resistant cells and adagrasib resistant cells were determined to be A59S and Y96D mutations.

### 1.3 Determination of Cross Resistance of Secondary Mutations Developed Against Sotorasib or Adagrasib

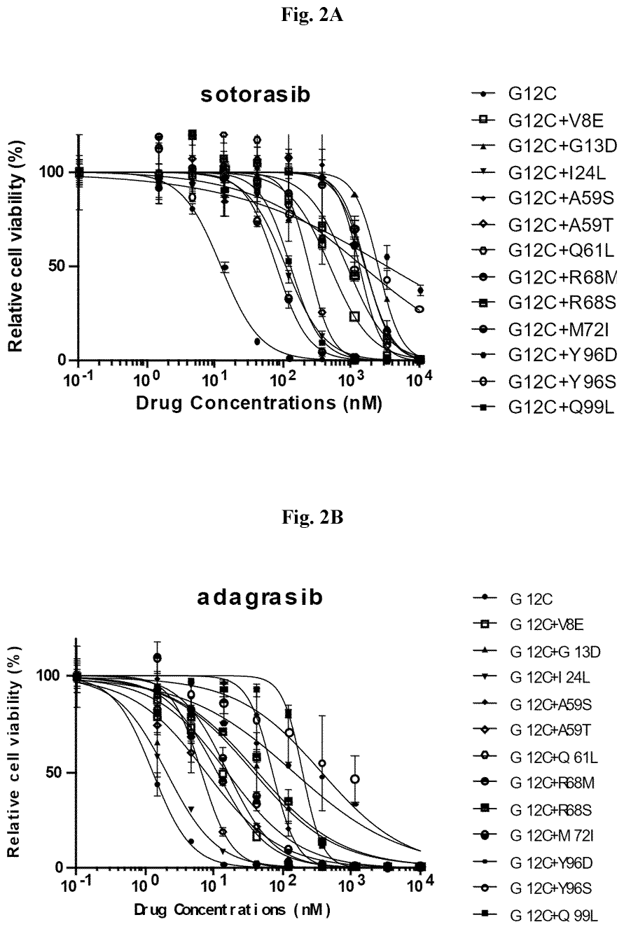

To evaluate the cross resistance, growth inhibition assays with sotorasib and adagrasib were conducted for resistant clones generated by the ENU mutagenesis. The FIG. 2A shows the growth inhibition curves of Ba/F3 cells harboring KRAS G12C and secondary mutations derived through ENU mutagenesis and treated with the indicated concentrations of sotorasib for 72 hours. The results are shown as the mean values of three individual experiments. Error bars indicate the standard deviation (SD). The FIG. 2B shows the growth inhibition curves of Ba/F3 cells harboring KRAS G12C and secondary mutations derived through ENU mutagenesis and treated with the indicated concentrations of adagrasib for 72 hours.

Because the IC₅₀ values of sotorasib and adagrasib for KRAS G12C parental Ba/F3 cells were different, 12.4 nM and 1.3 nM, respectively, the resistance index (RI) was used to clearly compare the degree of resistance of each secondary mutation. RI was defined as a ratio of the IC₅₀ of each drug for each resistant clone to the respective IC₅₀ of each drug for the parental Ba/F3 cells with KRAS G12C.

The table 1 summarises the resistance index (RI) values of sotorasib- and adagrasib-resistant clones generated through ENU mutagenesis. The RI value was calculated by the following formula: IC₅₀ of each resistant clone/IC₅₀ of parental Ba/F3 cell with KRAS G12C. In the table 1, low resistance corresponds to an RI value less than 10, moderate resistance corresponds to an RI value higher than 10 but lower than 100, and high resistance corresponds to an RI value higher than 100.

TABLE 1
resistance index (RI) values

| | sotorasib | adagrasib |
|---|---|---|
| G12C | 1 | 1 |
| G12C + V8E | 39.5 | 8.7 |
| G12C + G13D | 202 | 28.2 |
| G12C + I24L | 8.9 | 1.6 |
| G12C + A59S | 131 | 54.8 |
| G12C + A59T | 107 | 6.0 |
| G12C + Q61L | 19.6 | 5.1 |
| G12C + R68M | 129 | 12.2 |
| G12C + R68S | 71.1 | 32.5 |
| G12C + M72I | 6.5 | 12.2 |
| G12C + Y96D | 268 | 111 |
| G12C + Y96S | 112 | 286 |
| G12C + Q99L | 10.4 | 150 |

As can be taken from table 1, for sotorasib, G13D, A59S/T, R68M and Y96D/S were highly resistant (RI>100), while for adagrasib, Y96D/S and Q99L were highly resistant.

The spectrum of the secondary mutations was significantly different between sotorasib and adagrasib resistant clones. For example, G13D, A59S/T and R68M conferred resistance to sotorasib but remained sensitive to adagrasib, while a Q99L secondary mutation was resistant to adagrasib but was sensitive to sotorasib.

The model of Ba/F3 cells subjected to ENU mutagenesis provides secondary KRAS mutations showing resistance to the KRAS G12C inhibitors sotorasib or adagrasib. This method is efficient in generating resistant clones with secondary mutations, although it is artificial and it is known that there is preference of G:C to A:T transitions and A:T to T:A transversions, and A:T to G:C base changes, as was seen here. Nevertheless, the secondary mutations identified in this assay are consistent with the acquired resistance mutations found in clinical setting after EGFR-, ALK- and MET-TKIs treatment failure.

The results using this model however showed that Y96D/S secondary mutations were shared with both KRAS G12C inhibitors sotorasib and adagrasib as resistant mutations, indicating that the Y96D/S secondary mutations would cause cross-resistance to both sotorasib and adagrasib. Without wishing to be bound to any therory, it is assumed that Y96 is located at the entrance of the hydrophobic pocket where sotorasib and adagrasib bind with the KRAS G12C protein.

### Example 2

### Validation of KRAS Secondary Y96D and Y96S Mutations

2.1 Validation of KRAS Secondary Mutations that were Detected in Sotorasib or Adagrasib-Resistant Cells in Ba/F3 Cells

To confirm that observed resistance was due to the emergence of the secondary KRAS mutations and not due to unidentified mechanisms, KRAS G12C plus either of the seven secondary mutations G13D, A59S/T, R68M, Y96D/S, or Q99L with RI>100 as determined in Example 1 were introduced into Ba/F3 cells and growth inhibition assays using these Ba/F3 were then conducted.

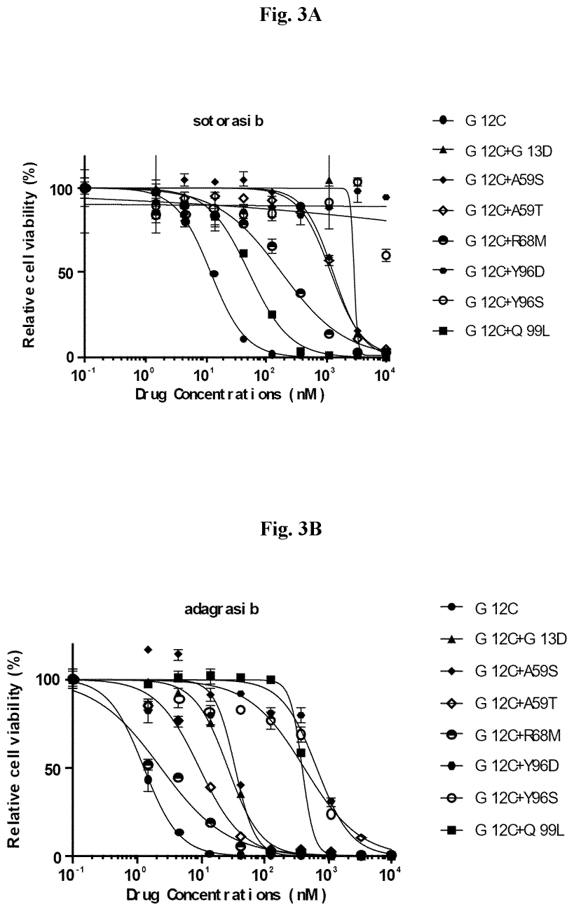

The FIG. 3A shows the growth inhibition assay of Ba/F3 cells harboring KRAS G12C plus secondary mutations and treated with the indicated concentrations of sotorasib for 72 hours. The results are shown as the mean values of three individual experiments. Error bars indicate the standard deviation (SD). The FIG. 3B shows the growth inhibition assay of Ba/F3 cells harboring KRAS G12C plus secondary mutations and treated with the indicated concentrations of adagrasib for 72 hours.

The table 2 summarises the the resistance index (RI) values of Ba/F3 cells harboring KRAS G12C plus secondary mutations. The RI value was calculated with the following formula: IC₅₀ of each reconstructed Ba/F3 cell/IC₅₀ of parental Ba/F3 cells with KRAS G12C. In the table 2, low resistance corresponds to an RI value less than 10, moderate resistance corresponds to an RI value higher than 10 but lower than 100, and high resistance corresponds to an RI value higher than 100.

TABLE 2
resistance index (RI) values

| | sotorasib | adagrasib |
|---|---|---|
| G12C | 1 | 1 |
| G12C + G13D | 238 | 21.0 |
| G12C + A59S | 107 | 27.3 |
| G12C + A59T | 97.2 | 7.5 |
| G12C + R68M | 15.3 | 1.8 |
| G12C + Y96D | >500 | >500 |
| G12C + Y96S | >500 | 333 |
| G12C + Q99L | 4.3 | 307 |

As can be taken from the table 2, while some of the resistant mutations such as G13S, A59S/T, R68M, Q99L could be overcome by switching the KRASG12C inhibitors from sotorasib to adagrasib or vice versa, Y96D/S mutations that emerged after treatment with high concentrations of sotorasib or adagrasib were highly resistant to both inhibitors.

2.2 Validation of KRAS Secondary Mutations that were Detected in Sotorasib or Adagrasib Resistant Cells in H358 Cells

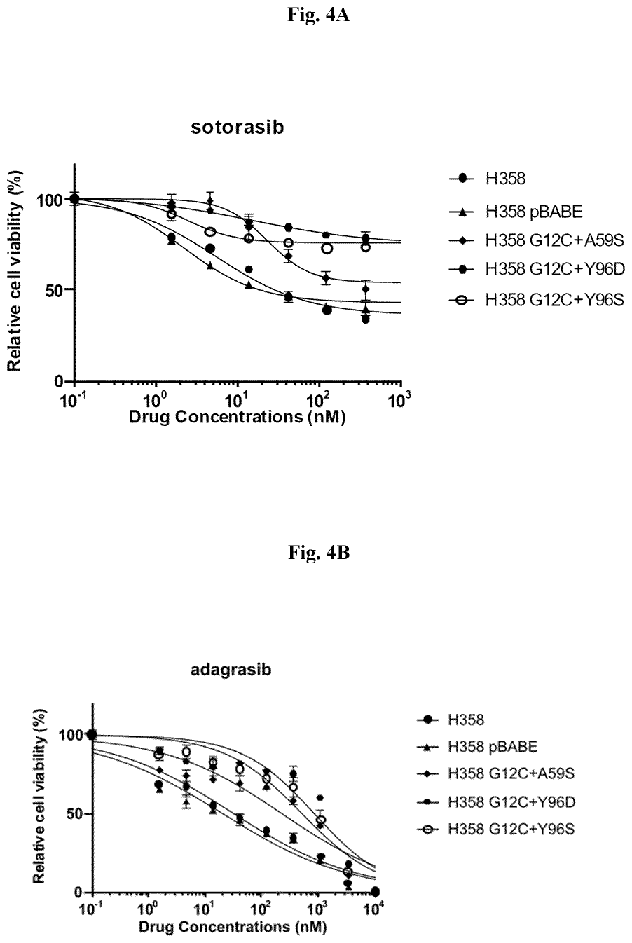

In further experiments, KRAS G12C plus Y96D, G12C plus Y96S and G12C plus A59S were retrovirally introduced into NCI-H358 cells that originally harbored KRAS G12C mutation. The FIG. 4A shows the growth inhibition assay of H358 cells with KRAS G12C plus secondary mutations treated with the indicated concentrations of sotorasib for 72 hours. The results are shown as the mean values of three individual experiments. Error bars indicate the standard deviation (SD). The FIG. 4B shows the growth inhibition assay of H358 cells with KRAS G12C plus secondary mutations treated with the indicated concentrations of adagrasib for 72 hours. As can be taken from FIGS. 4A and 4B, in the growth inhibition assay, H358 cells harboring G12C plus Y96D or G12C plus Y96S showed approximately 30 times higher IC50 values compared to parental H358 cells.

This demonstrates that H358 cells harboring G12C plus A59S were less resistant to both of KRASG12C inhibitors than H358 cells harboring G12C plus Y96D or G12C plus Y96S, consistent with the results in Ba/F3 cells.

### Example 3

Determination of Effects of SOS1 Inhibitor BI-3406 and BI-3406 in Combination with MEK Inhibitor Trametinib to Overcome Resistance to KRAS G12C Inhibitors by Secondary Mutations Y96D and Y96S

### 3.1 Determination of Effects of SOS1 Inhibitor BI-3406 in Ba/F3 Cells

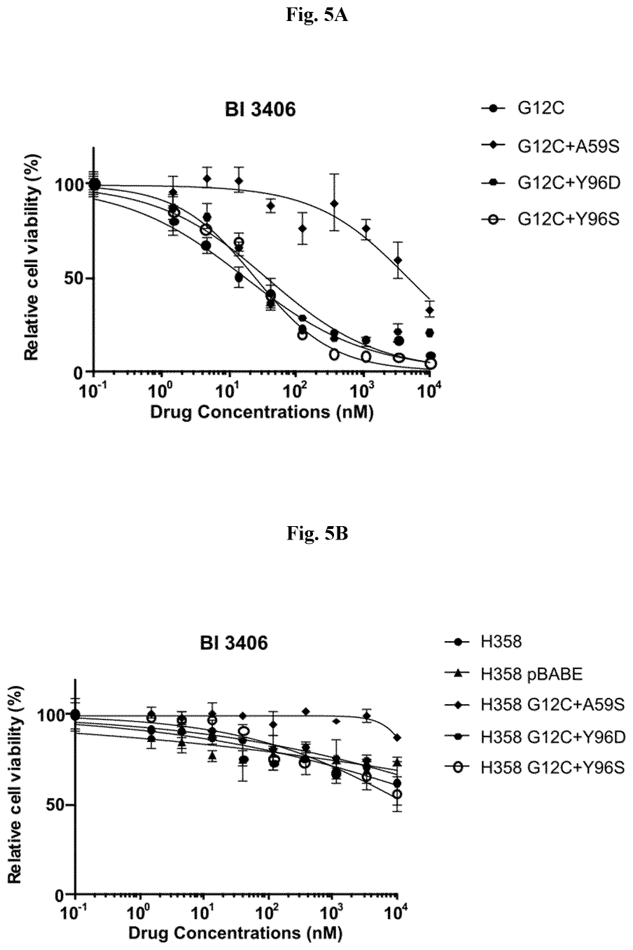

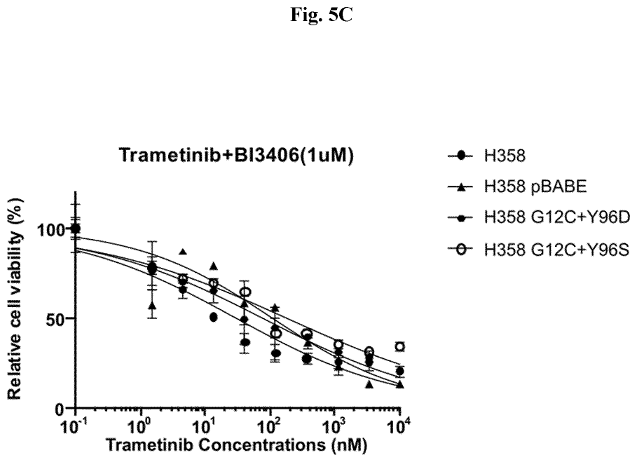

Ba/F3 cells harboring G12C plus either, A59S, Y96D or Y96S were incubated with SOS1 inhibitor BI-3406 or TNO 155, a SHP2 inhibitor, as described above. The FIG. 5A shows the growth inhibition assay of Ba/F3 cells harboring KRAS G12C plus secondary A59S, Y96D or Y96S mutation treated with the indicated concentrations of BI-3406 for 72 hours. The results are shown as the mean values of three individual experiments. Error bars indicate the standard deviation (SD).

The following table 3 summarises the IC50 values of Ba/F3 cells harboring KRAS G12C plus secondary A59S, Y96D or Y96S mutation treated with BI-3406 or TNO 155 for 72 hours.

TABLE 3
IC₅₀ values of Ba/F3 cells harboring KRAS G12C plus secondary
A59S, Y96D or Y96S mutation treated with BI-3406 or TNO 155

| Ba/F3 | BI-3406 [nM] | TNO 155 [nM] |
|---|---|---|
| G12C | 18.3 | 455 |
| G12C + A59S | 4850 | 3050 |
| G12C + Y96D | 38 | 1086 |
| G12C + Y96S | 25.7 | 1149 |

Treatment of Ba/F3 cells harboring G12C plus either Y96D or Y96S with BI-3406 resulted in an IC50 of 38.0 nM and 25.7 nM, while IC50 values of TNO 155 to Ba/F3 cells harboring G12C plus secondary mutations were higher than 1000 nM. In G12C plus A59S mutant Ba/F3 cells, neither BI-3406 nor TNO155 was able to reduce tumor cell proliferation, showing an IC50 of 4850 nM and 3050 nM, respectively.

These results show that the SOS1 inhibitor BI-3406 showed activity against an in vitro model with concurrent G12C and secondary Y96D or Y96S mutations.

3.2 Determination of Effects of SOS1 Inhibitor BI-3406 Alone and in Combination with MEK Inhibitor Trametinib in H358 Cells Harboring KRAS G12C and Y96D or Y96S Mutations

In 2D- and 3D-growth inhibition assays H358 cells expressing G12C plus Y96D or G12C plus Y96S were incubated with either SOS1 inhibitor BI-3406 alone or BI-3406 in combination with MEK inhibitor trametinib as described above.

The FIG. 5B shows the three-dimensional (3D) growth inhibition assay of H358 cells with KRAS G12C plus secondary A59S, Y96D or Y96S mutation treated with the indicated concentrations of BI-3406 for 72 hours. The following table 4 summarises the IC₅₀ and IC₅₀* values of isogenic H358 cells with KRAS G12C plus secondary mutations A59S, Y96D and Y96S treated with different concentrations of BI-3406 alone for 72 hours in the 2D-assay and in the 3D-assay as illustrated in FIG. 5B. The IC₅₀* value indicates the concentrations that gave the response half way between maximal and minimal inhibition.

TABLE 4
IC₅₀ and IC₅₀* values of H358 cells with KRAS G12C plus A59S,
Y96D and Y96S mutations treated with SOS1 inhibitor BI-3406

BI-3406 [nM]

| H358 isogenic | 2D | 2D | 3D | 3D |
|---|---|---|---|---|
| cells | IC₅₀ | IC₅₀* | IC₅₀ | IC₅₀* |
| H358 | >10000 | 63.3 | >10000 | 25.9 |
| pBABE-puro | >10000 | 6.5 | >10000 | 1.9 |
| G12C + A59S | >10000 | >10000 | >10000 | >10000 |
| G12C + Y96D | >10000 | 1606 | >10000 | 967.1 |
| G12C + Y96S | >10000 | 194.4 | >10000 | 117.3 |

As can be taken from Table 4 and FIG. 5B, in both 2D and 3D assays BI-3406 monotherapy modestly inhibited growth of H358 parental cells as well as the growth of H358 cells plus secondary Y96D/S mutation.

The FIG. 5C shows the three-dimensional (3D) growth inhibition assay of H358 cells with KRAS G12C plus secondary Y96D or Y96S mutation treated with a combination of 1 μM SOS1 inhibitor BI-3406 and indicated concentrations of trametinib for 72 hours. The table 5 summarises the IC₅₀ values of isogenic H358 cells with KRAS G12C plus secondary mutations Y96D and Y96S treated with a combination of SOS1 inhibitor BI-3406 and MEK inhibitor trametinib for 72 hours in the 3D-assay as illustrated in FIG. 5C.

TABLE 5
IC₅₀ values of H358 cells with KRAS G12C plus
Y96D and Y96S mutations treated with SOS1 inhibitor
BI-3406 and MEK inhibitor trametinib

| | BI-3406 + |
|---|---|
| H358 isogenic | trametinib [nM] |
| cells | 3D |
| H358 | 28.7 |
| pBABE-puro | 106.2 |
| G12C + Y96D | 83.3 |
| G12C + Y96S | 182.7 |

As can be taken from table 5 and FIG. 5C, H358 cells with G12C plus secondary Y96D/S mutations were sensitive to a treatment with a combination of SOS1 inhibitor BI-3406 plus MEK inhibitor trametinib, and their sensitivities were comparable with parental H358 cells.

These results show that the SOS1 inhibitor BI-3406 alone or in combination with the MEK inhibitor trametinib showed potent inhibitory activity in Ba/F3 and H358 cells carrying a KRAS G12C mutation and a secondary Y96D/S mutation. This provides an option for switching to a second-line therapy to overcome an acquired resistance caused by secondary KRAS mutations as are expected to emerge upon acquiring resistance in the course of treatment with sotorasib and adagrasib.

1. A method of treating and/or preventing cancer, the method comprising administering to a patient a therapeutically effective amount of a SOS1 inhibitor or a therapeutically effective amount of a MEK inhibitor or a therapeutically effective amount of a SOS1 inhibitor in combination with a therapeutically effective amount of a MEK inhibitor, wherein the cancer is resistant to treatment with an inhibitor of KRAS G12C.

2. The method according to claim 1, wherein the cancer is resistant to treatment with sotorasib (AMG 510) and/or adagrasib (MRTX 849).

3. The method according to claim 1, wherein the cancer cells of the cancer exhibit a primary KRAS G12C mutation and further exhibit a secondary mutation Y96X.

4. The method according to claim 1, wherein the SOS1 inhibitor is selected from the group consisting of the following compounds and the pharmaceutically acceptable salts thereof:

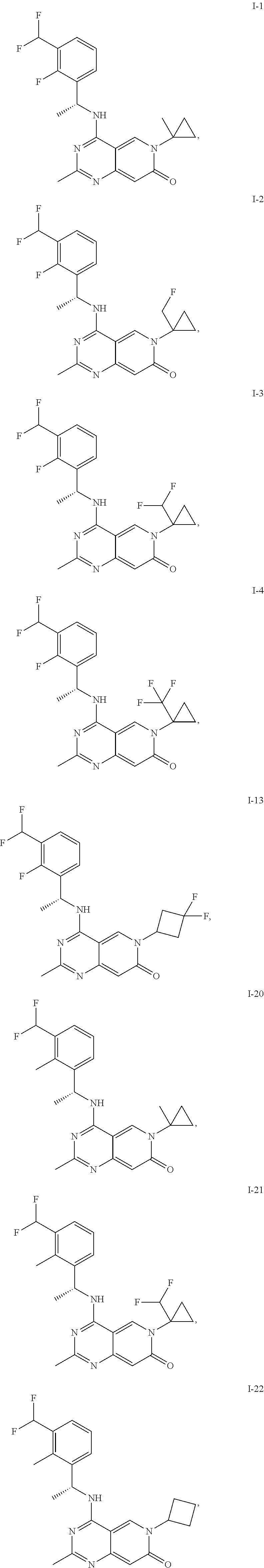

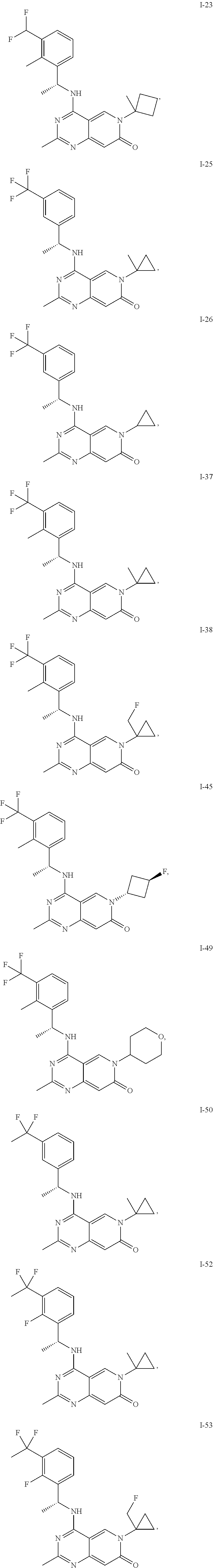

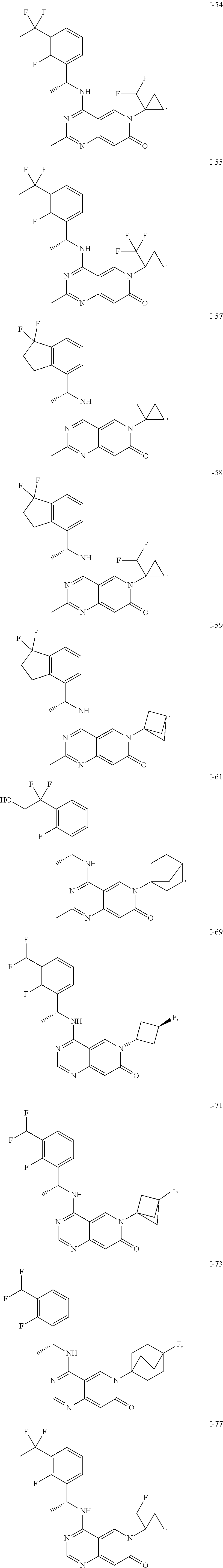

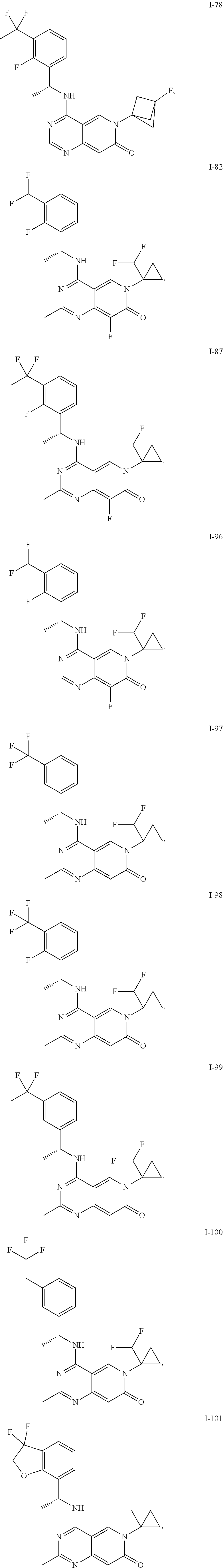

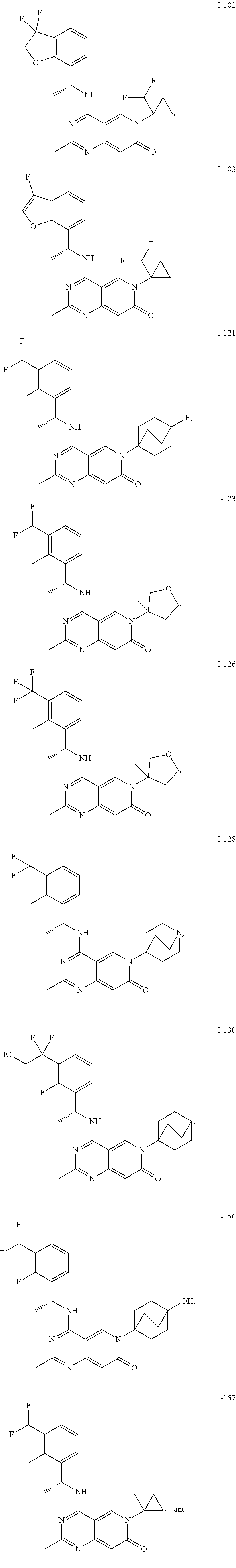

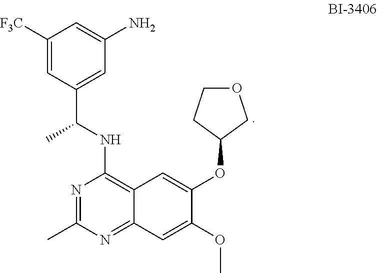 [SMILES: COc1cc2nc(C)nc(N[C@H](C)c3cc(N)cc(C(F)(F)F)c3)c2cc1O[C@H]1CCOC1]

5. The method according to claim 1, wherein the MEK inhibitor is selected from the group consisting of trametinib, cobimetinib, binimetinib, selumetinib, refametinib and the following compounds, and the pharmaceutically acceptable salts thereof:

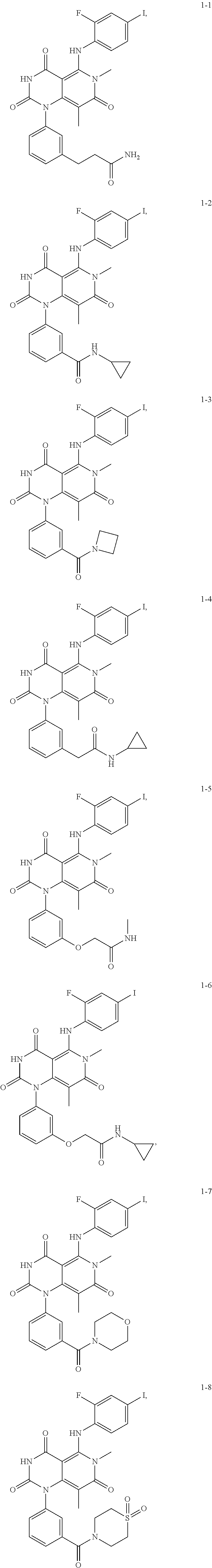

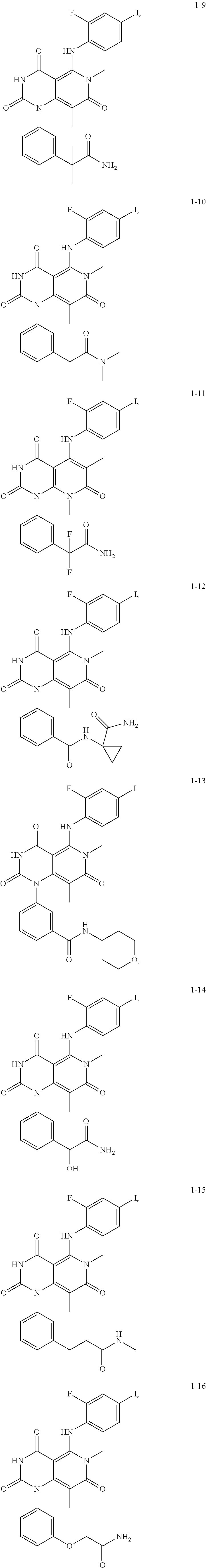

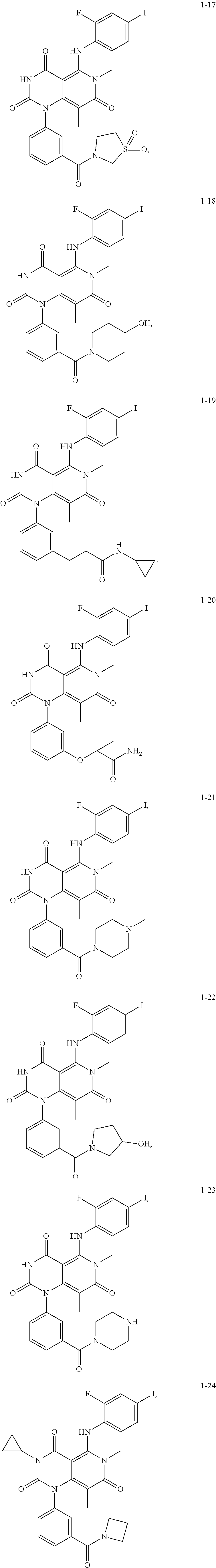

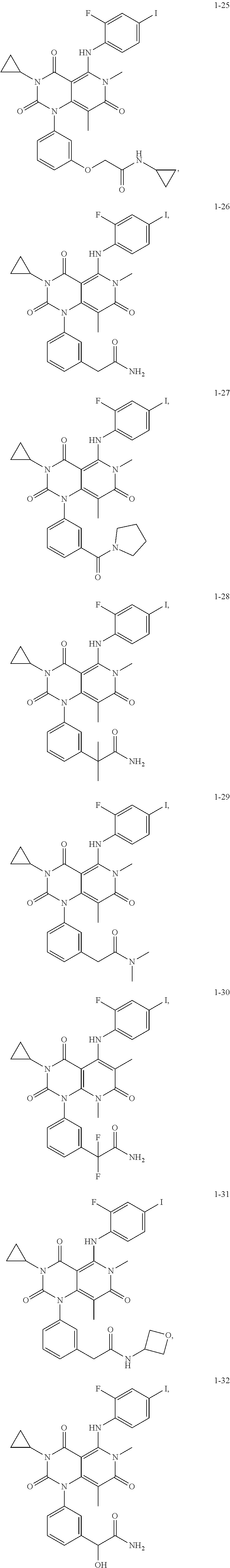

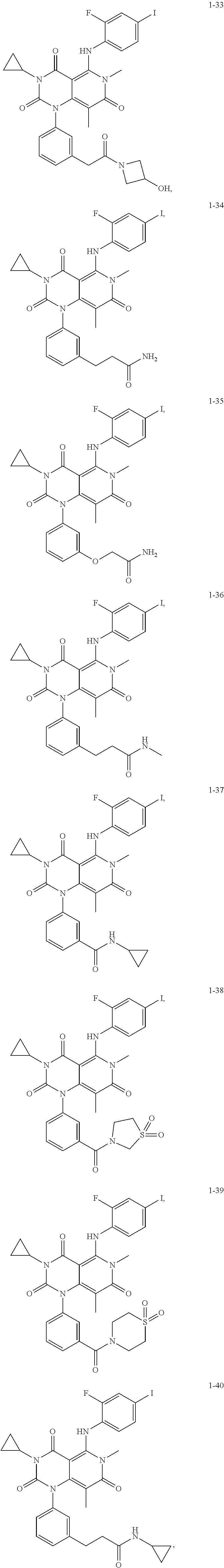

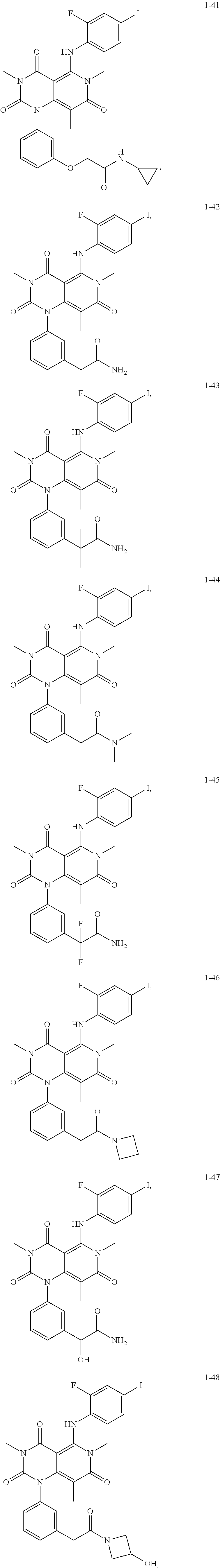

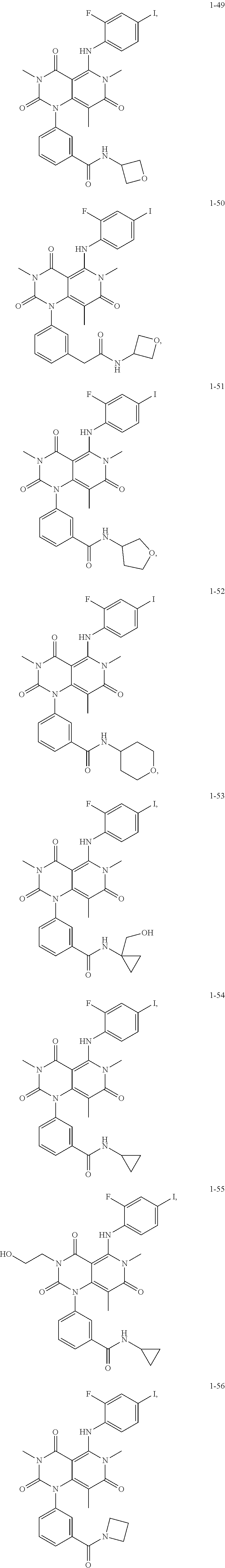

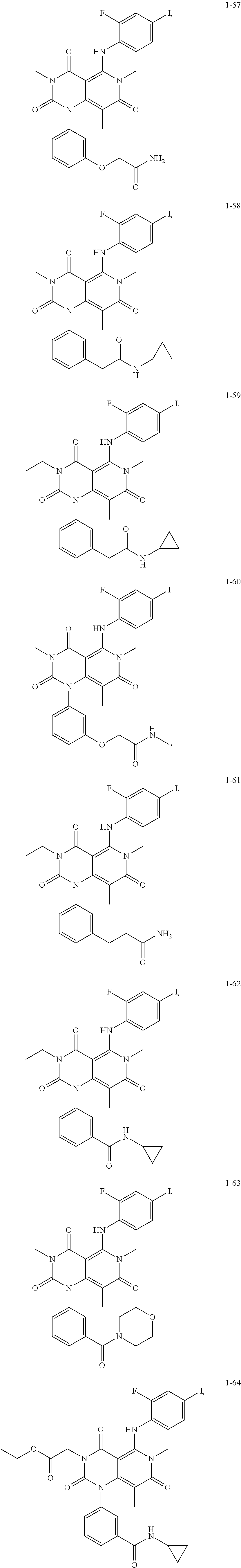

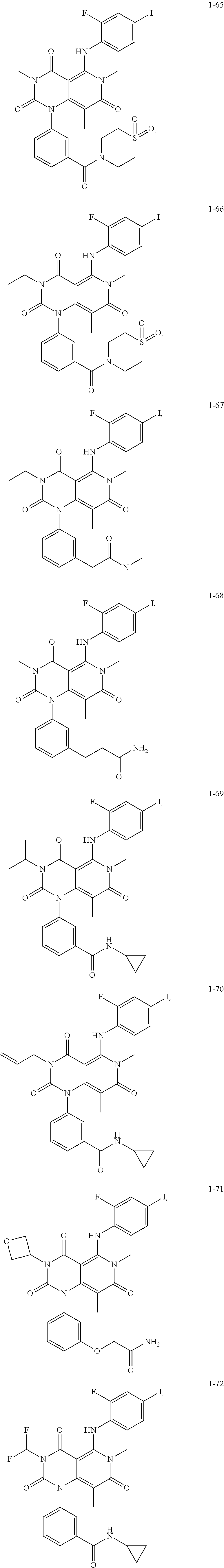

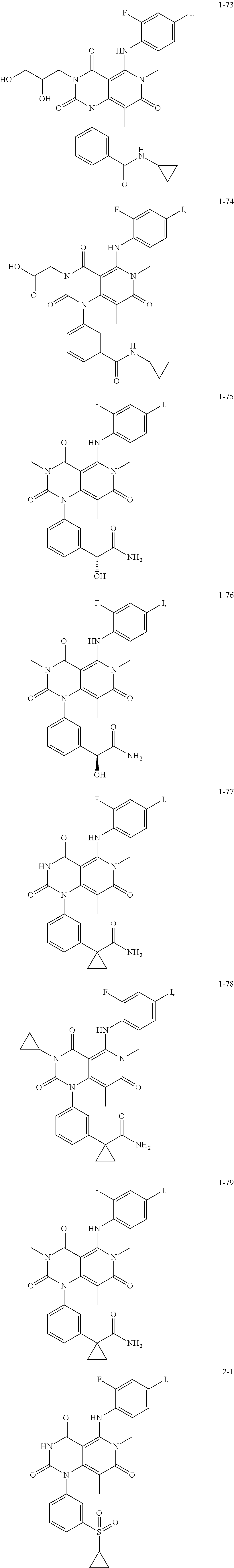

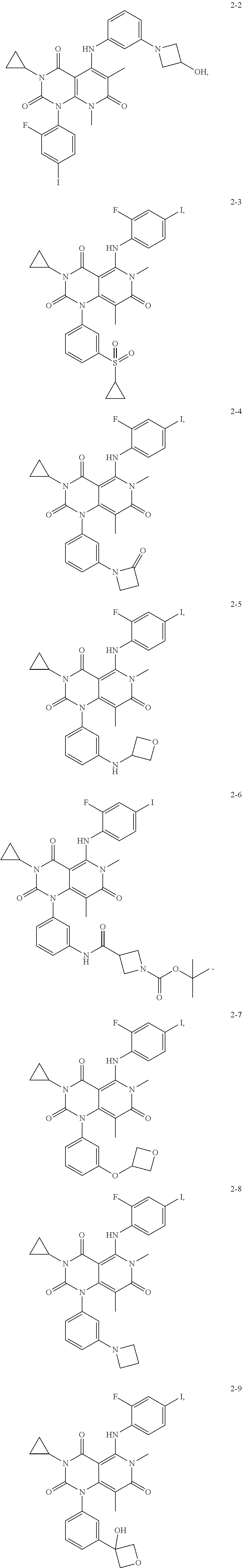

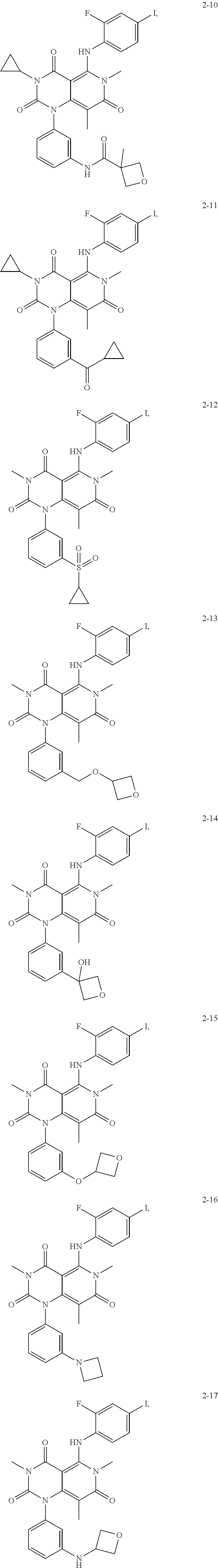

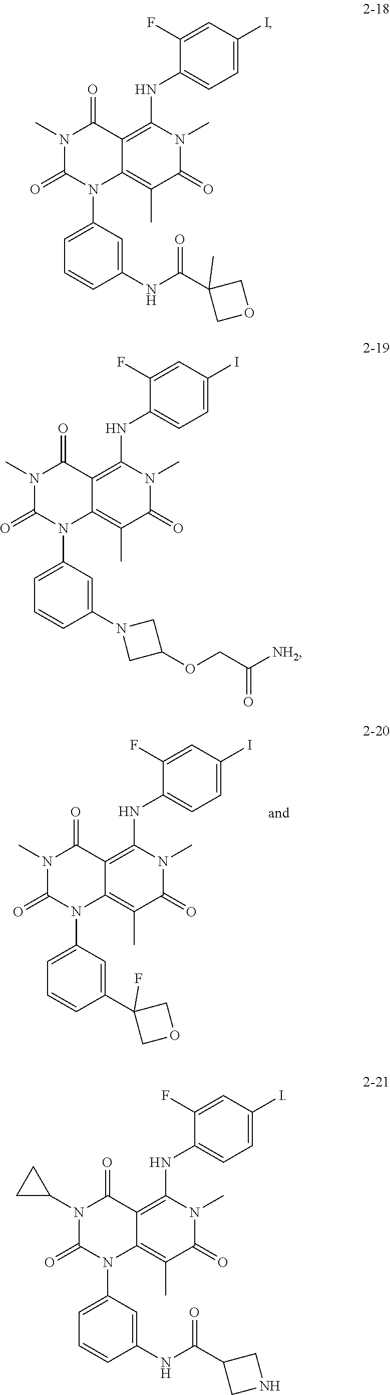

6. The method according to claim 1, wherein the cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

7. A method according to claim 1, wherein the SOS1 inhibitor is administered as a pharmaceutical composition comprising as active ingredient an SOS1 inhibitor, wherein the MEK inhibitor is administered as a pharmaceutical composition comprising as active ingredient an MEK inhibitor, and the combination of the SOS1 inhibitor and the MEK inhibitor is administered as a pharmaceutical composition comprising as active ingredients an SOS1 inhibitor and an MEK inhibitor.

8. A kit comprising in one or more containers:
(i) a first pharmaceutical composition or dosage form comprising an SOS1 inhibitor, and, optionally, pharmaceutically acceptable carriers, excipients and/or vehicles; and/or
(ii) a second pharmaceutical composition or dosage form comprising an MEK inhibitor, and, optionally, pharmaceutically acceptable carriers, excipients and/or vehicles; and
(iii) optionally a package insert comprising instructions,
said instructions indicating use in the treatment and/or prevention of cancer, wherein the cancer is resistant to treatment with an inhibitor of KRAS G12C.

9. (canceled)

10. (canceled)

11. (canceled)

12. (canceled)

13. (canceled)

14. (canceled)

15. An in vitro method for detecting or diagnosing that an individual has acquired resistance to treatment with an inhibitor of KRAS G12C and/or is susceptible to treatment with a SOS1 inhibitor or a MEK inhibitor or a combination of a SOS1 inhibitor and a MEK inhibitor, the method comprising the step of:
determining, in a biological sample from an individual, the presence of a KRAS mutation Y96X, wherein the individual is classified as being resistant to treatment with an inhibitor of KRAS G12C and being susceptible to treatment with a SOS1 inhibitor or a MEK inhibitor or a combination of a SOS1 inhibitor and a MEK inhibitor, based on the detection of the KRAS mutation Y96X.

16. (canceled)

17. The method according to claim 3, wherein the secondary mutation Y96X is selected from the group consisting of Y96D and Y96S.

18. The in vitro method according to claim 15, wherein the KRAS mutation Y96X is selected from the group consisting of Y96D and Y96S.
